SEQUENCE LISTING (23 sequences)

SEQ ID NO: 1 - DNA, 2005 bp, human
atggaggggaacaagctggaggagcaggactctagccctccacagtccactccagggctcatgaaggggaacaagcgtgaggagcaggggctgggccccgaacctgcggcgccagcagcccacggcggaggaggaggccctgatcgagttccaccgctcctaccgagagctcttcgagttcttctgcaacaacaccaccatccacggcgccatccgcctggtgtgctcccagcacaaccgcatgaagacggccttctgggcagtgctgtggctctgcacctttggcatgatgtactggcaattcggcctgcttttcggagagtacttcagctaccccgtcagcctcaacatcaacctcaactcgacaagctcgtcttccccgcagtgaccatctgcaccctcaatccctacaggtacccggaaattaaagaggagcttgaggagctggaccgcatcacagagcagacgctctttgacctgtacaaatacagctccttcaccactctcgtggccggctccgcagccgtcgcgacctgcgggggactctgccgcaccccttgcagcgcctgagggtcccgccccgcctcacggggcccgtcgagcccgtagcgtggcctccagcttgcgggacaacaacccccaggtggactggaaggactggaagatcggcttccagctgtgcaaccagaacaaatcggactgcttctaccagacatactcatcaggggtggatgcggtgagggagtggtaccgcttccactacatcaacatcctgtcgaggctgccagagactctgccatccctggaggaggacacgctgggcaacttcatcttcgcctgccgcttcaaccaggtctcctgcaaccaggcgaattactctcacttccaccacccgatgtatggaaactgctatactttcaatgacaagaacaactccaacctctggatgtcttccatgcctggaatcaacaacggtctgtccctgatgctgcgcgcagagcagaatgacttcattcccctgctgtccacagtgactggggcccgggtaatggtgcacgggcaggatgaacctgcctttatggatgatggtggctttaacttgcggcctggcgtggagacctccatcagcatgaggaaggaaaccctggacagacttgggggcgattatggcgactgcaccaagaatggcagtgatgttcctgttgagaacctttacccttcaaagtacacacagcaggtgtgtattcactcctgcttccaggagagcatgatcaaggagtgtggctgtgcctacatcttctatccgcggccccagaacgtggagtactgtgactacagaaagcacagttcctgggggtactgctactataagctccaggttgacttctcctcagaccacctgggctgtttcaccaagtgccggaagccatgcagcgtgaccagctaccagctctctgctggttactcacgatggccctcggtgacatcccaggaatgggtcttccagatgctatcgcgacagaacaattacaccgtcaacaacaagagaaatggagtggccaaagtcaacatcttcttcaaggagctgaactacaaaaccaattctgagtctccctctgtcacgatggtcaccctcctgtccaacctgggcagccagtggagcctgtggttcggctcctcggtgttgtctgtggtggagatggctgagctcgtctttgacctgctggtcatcatgttcctcatgctgctccgaaggttccgaagccgatactggtctccaggccgagggggcaggggtgctcaggaggtagcctccaccctggcatcctcccctccttcccacttctgcccccaccccatgtctctgtccttgtcccagccaggccctgctccctctccagccttgacagcccctccccctgcctatgccaccctgggcccccgcccatctccagggggctctgcaggggccagttcctccacctgtcctctgggggggccctga

SEQ ID NO: 2 - DNA, 1923 bp, human
atgcacgtgaagaagtacctgctgaagggcctgcatcggctgcagaagggccccggctacacgtacaaggagctgctggtgtggtactgcgacaacaccaacacccacggccccaagcgcatcatctgtgaggggcccaagaagaaagccatgtggttcctgctcaccctgctcttcgccgccctcgtctgctggcagtggggcatcttcatcaggacctacttgagctgggaggtcagcgtctccctctccgtaggcttcaagaccatggacttccccgccgtcaccatctgcaatgctagccccttcaagtattccaaaatcaagcatttgctgaaggacctggatgagctgatggaagctgtcctggagagaatcctggctcctgagctaagccatgccaatgccaccaggaacctgaacttctccatctggaaccacacacccctggtccttattgatgaacggaacccccaccaccccatggtccttgatctctttggagacaaccacaatggcttaacaagcagctcagcatcagaaaagatctgtaatgcccacgggtgcaaaatggccatgagactatgtagcctcaacaggacccagtgtaccttccggaacttcaccagtgctacccaggcattgacagagtggtacatcctgcaggccaccaacatctttgcacaggtgccacagcaggagctagtagagatgagctaccccggcgagcagatgatcctggcctgcctattcggagctgagccctgcaactaccggaacttcacgtccatcttctaccctcactatggcaactgttacatcttcaactggggcatgacagagaaggcacttccttcggccaaccctggaactgaattcggcctgaagttgatcctggacataggccaggaagactacgtccccttccttgcgtccacggccggggtcaggctgatgcttcacgagcagaggtcataccccttcatcagagatgagggcatctacgccatgtcggggacagagacgtccatcggggtactcgtggacaagcttcagcgcatgggggagccctacagcccgtgcaccgtgaatggttctgaggtccccgtccaaaacttctacagtgactacaacacgacctactccatccaggcctgtcttcgctcctgcttccaagaccacatgatccgtaactgcaactgtggccactacctgtacccactgccccgtggggagaaatactgcaacaaccgggacttcccagactgggcccattgctactcagatctacagatgagcgtggcgcagagagagacctgcattggcatgtgcaaggagtcctgcaatgacacccagtacaagatgaccatctccatggctgactggccttctgaggcctccgaggactggattttccacgtcttgtctcaggagcgggaccaaagcaccaatatcaccctgagcaggaagggaattgtcaagctcaacatctacttccaagaatttaactatcgcaccattgaagaatcagcagccaataacatcgtctggctgctctcgaatctgggtggccagtttggcttctggatggggggctctgtgctgtgcctcatcgagtttggggagatcatcatcgactttgtgtggatcaccatcatcaagctggtggccttggccaagagcctacggcagcggcgagcccaagccagctacgctggcccaccgcccaccgtggccgagctggtggaggcccacaccaactttggcttccagcctgacacggccccccgcagccccaacactgggccctaccccagtgagcaggccctgcccatcccaggcaccccgccccccaactatgactccctgcgtctgcagccgctggacgtcatcgagtctgacagtgagggtgatgccatctaa

SEQ ID NO: 3 - DNA, 1950 bp, human
atggcacccggagagaagatcaaagccaaaatcaagaagaatctgcccgtgacgggccctcaggcgccgaccattaaagagctgatgcggtggtactgcctcaacaccaacacccatggctgtcgccgcatcgtggtgtcccgcggccgtctgcgccgcctcctctggatcgggttcacactgactgccgtggccctcatcctctggcagtgcgccctcctcgtcttctccttctatactgtctcagtttccatcaaagtccacttccggaagctggattttcctgcagtcaccatctgcaacatcaacccctacaagtacagcaccgttcgccaccttctagctgacttggaacaggagaccagagaggccctgaagtccctgtatggctttccagagtcccggaagcgccgagaggcggagtcctggaactccgtctcagagggaaagcagcctagattctcccaccggattccgctgctgatctttgatcaggatgagaagggcaaggccagggacttcttcacagggaggaagcggaaagtcggcggtagcatcattcacaaggcttcaaatgtcatgcacatcgagtccaagcaagtggtgggattccaactgtgctcaaatgacacctccgactgtgccacctacaccttcagctcgggaatcaatgccattcaggagtggtataagctacactacatgaacatcatggcacaggtgcctctggagaagaaaatcaacatgagctattctgctgaggagctgctggtgacctgcttctttgatggagtgtcctgtgatgccaggaatttcacgcttttccaccacccgatgcatgggaattgctatacattcaacaacagagaaaatgagaccattctcagcacctccatggggggcagcgaatatgggctgcaagtcattttgtacataaacgaagaggaatacaacccattcctcgtgtcctccactggagctaaggtgatcatccatcggcaggatgagtatcccttcgtcgaagatgtgggaacagagattgagacagcaatggtcacctctataggaatgcacctgacagagtccttcaagctgagtgagccctacagtcagtgcacggaggacgggagtgacgtgccaatcaggaacatctacaacgctgcctactcgctccagatctgccttcattcatgcttccagacaaagatggtggagaaatgtgggtgtgcccagtacagccagcctctacctcctgcagccaactactgcaactaccagcagcaccccaactggatgtattgttactaccaactgcatcgagcctttgtccaggaagagctgggctgccagtctgtgtgcaaggaagcctgcagctttaaagagtggacactaaccacaagcctggcacaatggccatctgtggtttcggagaagtggttgctgcctgttctcacttgggaccaaggccggcaagtaaacaaaaagctcaacaagacagacttggccaaactcttgatattctacaaagacctgaaccagagatccatcatggagagcccagccaacagtattgagatgcttctgtccaacttcggtggccagctgggcctgtggatgagctgctctgttgtctgcgtcatcgagatcatcgaggtcttcttcattgacttcttctctatcattgcccgccgccagtggcagaaagccaaggagtggtgggcctggaaacaggctcccccatgtccagaagctccccgtagcccacagggccaggacaatccagccctggatatagacgatgacctacccactttcaactctgctttgcacctgcctccagccctaggaacccaagtgcccggcacaccgccccccaaatacaataccttgcgcttggagagggccttttccaaccagctcacagatacccagatgctagatgagctctga

SEQ ID NO: 4 - protein, 668 aa, human
MEGNKLEEQDSSPPQSTPGLMKGNKREEQGLGPEPAAPQQPTAEEEALIEFHRSYRELFEFFCNNTTIHGAIRLVCSQHNRMKTAFWAVLWLCTFGMMYWQFGLLFGEYFSYPVSLNINLNSDKLVFPAVTICTLNPYRYPEIKEELEELDRITEQTLFDLYKYSSFTTLVAGSRSRRDLRGTLPHPLQRLRVPPPPHGARRARSVASSLRDNNPQVDWKDWKIGFQLCNQNKSDCFYQTYSSGVDAVREWYRFHYINILSRLPETLPSLEEDTLGNFIFACRFNQVSCNQANYSHFHHPMYGNCYTFNDKNNSNLWMSSMPGINNGLSLMLRAEQNDFIPLLSTVTGARVMVHGQDEPAFMDDGGFNLRPGVETSISMRKETLDRLGGDYGDCTKNGSDVPVENLYPSKYTQQVCIHSCFQESMIKECGCAYIFYPRPQNVEYCDYRKHSSWGYCYYKLQVDFSSDHLGCFTKCRKPCSVTSYQLSAGYSRWPSVTSQEWVFQMLSRQNNYTVNNKRNGVAKVNIFFKELNYKTNSESPSVTMVTLLSNLGSQWSLWFGSSVLSWEMAELVFDLLVIMFLMLLRRFRSRYWSPGRGGRGAQEVASTLASSPPSHFCPHPMSLSLSQPGPAPSPALTAPPPAYATLGPRPSPGGSAGASSSTCPLGGP

SEQ ID NO: 5 - protein, 640 aa, human
MHVKKYLLKGLHRLQKGPGYTYKELLVWYCDNTNTHGPKRIICEGPKKKAMWFLLTLLFAALVCWQWGIFIRTYLSWEVSVSLSVGFKTMDFPAVTICNASPFKYSKIKHLLKDLDELMEAVLERILAPELSHANATRNLNFSIWNHTPLVLIDERNPHHPMVLDLFGDNHNGLTSSSASEKICNAHGCKMAMRLCSLNRTQCTFRNFTSATQALTEWYILQATNIFAQVPQQELVEMSYPGEQMILACLFGAEPCNYRNFTSIFYPHYGNCYIFNWGMTEKALPSANPGTEFGLKLILDIGQEDYVPFLASTAGVRLMLHEQRSYPFIRDEGIYAMSGTETSIGVLVDKLQRMGEPYSPCTVNGSEVPVQNFYSDYNTTYSIQACLRSCFQDHMIRNCNCGHYLYPLPRGEKYCNNRDFPDWAHCYSDLQMSVAQRETCIGMCKESCNDTQYKMTISMADWPSEASEDWIFHVLSQERDQSTNITLSRKGIVKLNIYFQEFNYRTIEESAANNIVWLLSNLGGQFGFWMGGSVLCLIEFGEIIIDFVWITIIKLVALAKSLRQRRAQASYAGPPPTVAELVEAHTNFGFQPDTAPRSPNTGPYPSEQALPIPGTPPPNYDSLRLQPLDVIESDSEGDAI

SEQ ID NO: 6 - protein, 647 aa, human
MAPGEKIKAKIKKNLPVTGPQAPTIKELMRWYCLNTNTHGCRRIVVSRGRLRRLLWIGFTLTAVALILWQCALLVFSFYTVSVSIKVHFRKLDFPAVTICNINPYKYSTVRHLLADLEQETREALKSLYGFPESRKRREAESWNSVSEGKQPRFSHRIPLLIFDQDEKGKARDFFTGRKRKVGGSIIHKASNVMHIESKQVVGFQLCSNDTSDCATYTFSSGINAIQEWYKLHYMNIMAQVPLEKKINMSYSAEELLVTCFFDGVSCDARNFTLFHHPMHGNCYTFNNRENETILSTSMGGSEYGLQVILYINEEEYNPFLVSSTGAKVIIHRQDEYPFVEDVGTEIETAMVTSIGMHLTESFKLSEPYSQCTEDGSDVPIRNIYNAAYSLQICLHSCFQTKMVEKCGCAQYSQPLPPAANYCNYQQHPNWMYCYYQLHRAFVQEELGCQSVCKEACSFKEWTLTTSLAQWPSWSEKWLLPVLTWDQGRQVNKKLNKTDLAKLLIFYKDLNQRSIMESPANSIEMLLSNFGGQLGLWMSCSWCVIEIIEVFFIDFFSIIARRQWQKAKEWWAWKQAPPCPEAPRSPQGQDNPALDIDDDLPTFNSALHLPPALGTQVPGTPPPKYNTLRLERAFSNQLTDTQMLDEL

SEQ ID NO: 7 - DNA, 1917 bp, human
atggctgagcaccgaagcatggacgggagaatggaagcagccacacgggggggctctcacctccaggctgcagcccagacgccccccaggccggggccaccatcagcaccaccaccaccacccaaggaggggcaccaggaggggctggtggagctgcccgcctcgttccgggagctgctcaccttcttctgcaccaatgccaccatccacggcgccatccgcctggtctgctcccgcgggaaccggctcaagacgacgtcctgggggctgctgtccctgggagccctggtcgcgctctgctggcagctggggctcctctttgagcgtgactggcaccgcccggtcctcatggccgtctctgtgcactcggagcgcaagctggtcccgctggtcaccctgtgtgacgggaacccacgtcggccgagtccggtcctccgccatctggagctgctggacgagtttgccagggagaacattgactccctgtagaacgtcaacctcagcaaaggcagagccgccctctccgccactgtcccccgccacgagcccccgttccacctggaccgggagatccgtctgcagaggctgagccactcgggcagccgggtcagagtggggttcagactgtgcaacagcacgggcggcgactgcttttaccgaggctacacgtcaggcgtggcggctgtccaggactggtaccacttccactatgtggatatcctggccctgctgcgcgcggcatgggaggacagccacgggagccaggacggccacttcgtcctctcctgcagttacgatggcctggactgccaggcccgacagttccggaccttccaccaccccacctacggcagctgctacacggtcgatggcgtctggacagctcagcgccccggcatcacccacggagtcggcctggtcctcagggttgagcagcagcctcacctccctctgctgtccacgctggccggcatcagggtcatggttcacggccgtaaccacacgcccttcctggggcaccacagcttcagcgtccggccagggacggaggccaccatcagcatccgagaggacgaggtgcaccggctcgggagcccctacggccactgcaccgccggcggggaaggcgtggaggtggagctgctacacaacacctcctacaccaggcaggcctgcctggtgtcctgcttccagcagctgatggtggagacctgctcctgtggctactacctccaccctctgccggcgggggctgagtactgcagctctgcccggcaccctgcctggggacactgcttctaccgcctctaccaggacctggagacccaccggctcccctgtacctcccgctgccccaggccctgcagggagtctgcattcaagctctccactgggacctccaggtggccttccgccaagtcagctggatggactctggccacgctaggtgaacaggggctgccgcatcagagccacagacagaggagcagcctggccaaaatcaagatcgtctaccaggagctcaactaccgctcagtggaggaggcgcccgtgtactcggtgccgcagctgctctccgccatgggcagcctctacagcctgtggtttggggcctccgtcctctccctcctggagctcctggagctgctgctcgatgcttctgccctcaccctggtgctaggcggccgccggctccgcagggcgtggttctcctggcccagagccagccctgcctcaggggcgtccagctcaagccagaggccagtcagatgcccccgcctgcaggcggcacgtcagatgacccggagcccagcgggccttcatctcccacccgtcatgcttccagcggttctggcccgactctcagccgaagacagctgcgctgggccccagccccttgagactctggacacctga

SEQ ID NO: 8 - protein, 638 aa, human
MAEHRSMDGRMEAATRGGSHLQAAAQTPPRPGPPSAPPPPPKEGHQEGLVELPASFRELLTFFCTNATIHGAIRLVCSRGNRLKTTSWGLLSLGALVALCWQLGLLFERHWHRPVLMAVSVHSERKLLPLVTLCDGNPRRPSPVLRHLELLDEFARENIDSLYNVNLSKGRAALSATVPRHEPPFHLDREIRLQRLSHSGSRVRVGFRLCNSTGGDCFYRGYTSGVAAVQDWYHFHYVDILALLPAAWEDSHGSQDGHFVLSCSYDGLDCQARQFRTFHHPTYGSCYTVDGVWTAQRPGITHGVGLVLRVEQQPHLPLLSTLAGIRVMVHGRNHTPFLGHHSFSVRPGTEATISIREDEVHRLGSPYGHCTAGGEGVEVELLHNTSYTRQACLVSCFQQLMVETCSCGYYLHPLPAGAEYCSSARHPAWGHCFYRLYQDLETHRLPCTSRCPRPCRESAFKLSTGTSRWPSAKSAGWTLATLGEQGLPHQSHRQRSSLAKINIVYQELNYRSVEEAPVYSVPQLLSAMGSLYSLWFGASVLSLLELLELLLDASALTLVLGGRRLRRAWFSWPRASPASGASSIKPEASQMPPPAGGTSDDPEPSGPHLPRVMLPGVLAGVSAEESWAGPQPLETLDT

SEQ ID NO: 9 - DNA, 98 bp, artificial
  fragment of DNA
cctggggctattgctattataaactgcagggcgccttctccttggacagcctgggctgtttctccaagtgtcggaagccttgtagtgtgatcaactac

SEQ ID NO: 10 - DNA, 75 bp, artificial
  fragment DNA
cctgggggcgccttctccttggacagcctgggctgtctctccaagtgtcggaagccttgtagtgtgatcaactac

SEQ ID NO: 11 - DNA, 19 bp, artificial
  fragment DNA
cctgggtgtgaccaactac

SEQ ID NO: 12 - protein, 39 aa, artificial
  fragment protein
EFCDYRKQSSWGYCYYKLQGAFSLDSLGCFSKCRKPCSV

SEQ ID NO: 13 - protein, 29 aa, artificial
  fragment protein
EFCDYRKQSSWGRLLLGQPGLSLQVSEAL

SEQ ID NO: 14 - protein, 12 aa, artificial
  fragment protein
EFCDYRKQSSWV

SEQ ID NO: 15 - protein, 698 aa, rat
MLDHTRAPELNIDLDLHASNSPKGSMKGNQFKEQDPCPPQPMQGLGKGDKREEQGLGPEPSAPRQPTEEEEALIEFHRSYRELFQFFCNNTTIHGAIRLVCSKHNRMKTAFWAVLWLCTFGMMYWQFALLFEEYLSYPVSLNINLNSDKLVFPAVTVCTLNPYRYTEIKEELEELDRITEQTLFDLYKYNSSYTRQAGARRRSSRDLLGAFPHPLQRLRTPPPPYSGRTARSGSSSVRDNNPQVDRKDWKIGFQLCNQNKSDCFYQTYSSGVDAVREWYRFHYINILSRLSDTSPALEEEALGNFIFTCRFNQAPCNQANYSKFHHPMYGNCYTFNDKNNSNLWMSSMPGVNNGLSLTLRTEQNDFIPLLSTVTGARVMVHGQDEPAFMDDGGFNLRPGVETSISMRKEALDSLGGNYGDCTENGSDVPVKNLYPSKYTQQVCIHSCFQENMIKKCGCAYIFYPKPKGVEFCDYRKQSSWGYCYYKLQGAFSLDSLGCFSKCRKPCSVINYKLSAGYSRWPSVKSQDWIFEMLSLQNNYTINNKRNGVAKLNIFFKELNYKTNSESPSVTMVSLLSNLGSQWSLWFGSSVLSVVEMADVIFDLLVITLLMLLRRFRSRYWSPGRGARGAREVASTPASSFPSRFCPHPTSPPPSLPQQGMTPPLALTAPPPAYATLGPSAPPLDSAAPDCSACALAAL

SEQ ID NO: 16 - protein, 669 aa, human
MEGNKLEEQDSSPPQSTPGLMKGNKREEQGLGPEPAAPQQPTAEEEALIEFHRSYRELFEFFCNNTTIHGAIRLVCSQHNRMKTAFWAVLWLCTFGMMYWQFGLLFGEYFSYPVSLNINLNSDKLVFPAVTICTLNPYRYPEIKEELEELDRITEQTLFDLYKYSSFTTLVAGSRSRRDLRGTLPHPLQRLRVPPPPHGARRARSVASSLRDNNPQVDWKDWKIGFQLCNQNKSDCFYQTYSSGVDAVREWYRFHYINILSRLPETLPSLEEDTLGNFIFACRFNQVSCNQANYSHFHHPMYGNCYTFNDKNNSNLWMSSMPGINNGLSLMLRAEQNDFIPLLSTVTGARVMVHGQDEPAFMDDGGFNLRPGVETSISMRKETLDRLGGDYGDCTKNGSDVPVENLYPSKYTQQVCIHSCFQESMIKECGCAYIFYPRPQNVEYCDYRKHSSWGYCYYKLQVDFSSDHLGCFTKCRKPCSVTSYQLSAGYSRWPSVTSQEWVFQMLSRQNNYTVNNKRNGVAKVNIFFKELNYKTNSESPSVTMVTLLSNLGSQWSLWFGSSVLSVVEMAELVFDLLVIMFLMLLRRFRSRYWSPGRGGRGAQEVASTLASSPPSHFCPHPMSLSLSQPGPAPSPALTAPPPAYATLGPRPSPGGSAGASSSTCPLGGP

SEQ ID NO: 17 - DNA, 3151 bp, human
ccggccagcgggcgggctccccagccaggccgctgcacctgtcaggggaacaagctggaggagcaggaccctagacctctgcagcccataccaggtctcatggaggggaacaagctggaggagcaggactctagccctccacagtccactccagggctcatgaaggggaacaagcgtgaggagcaggggctgggccccgaacctgcggcgccccagcagcccacggcggaggaggaggccctgatcgagttccaccgctcctaccgagagctcttcgagttcttctgcaacaacaccaccatccacggcgccatccgcctggtgtgctcccagcacaaccgcatgaagacggccttctgggcagtgctgtggctctgcacctttggcatgatgtactggcaattcggcctgcttttcggagagtacttcagctaccccgtcagcctcaacatcaacctcaactcggacaagctcgtcttccccgcagtgaccatctgcaccctcaatccctacaggtacccggaaattaaagaggagctggaggagctggaccgcatcacagagcagacgctctttgacctgtacaaatacagctccttcaccactctcgtggccggctcccgcagccgtcgcgacctgcgggggactctgccgcaccccttgcagcgcctgagggtcccgcccccgcctcacggggcccgtcgagcccgtagcgtggcctccagcttgcgggacaacaacccccaggtggactggaaggactggaagatcggcttccagctgtgcaaccagaacaaatcggactgcttctaccagacatactcatcaggggtggatgcggtgagggagtggtaccgcttccactacatcaacatcctgtcgaggctgccagagactctgccatccctggaggaggacacgctgggcaacttcatcttcgcctgccgcttcaaccaggtctcctgcaaccaggcgaattactctcacttccaccacccgatgtatggaaactgctatactttcaatgacaagaacaactccaacctctggatgtcttccatgcctggaatcaacaacggtctgtccctgatgctgcgcgcagagcagaatgacttcattcccctgctgtccacagtgactggggcccgggtaatggtgcacgggcaggatgaacctgcctttatggatgatggtggctttaacttgcggcctggcgtggagacctccatcagcatgaggaaggaaaccctggacagacttgggggcgattatggcgactgcaccaagaatggcagtgatgttcctgttgagaacctttacccttcaaagtacacacagcaggtgtgtattcactcctgcttccaggagagcatgatcaaggagtgtggctgtgcctacatcttctatccgcggccccagaacgtggagtactgtgactacagaaagcacagttcctgggggtactgctactataagctccaggttgacttctcctcagaccacctgggctgtttcaccaagtgccggaagccatgcagcgtgaccagctaccagctctctgctggttactcacgatggccctcggtgacatcccaggaatgggtcttccagatgctatcgcgacagaacaattacaccgtcaacaacaagagaaatggagtggccaaagtcaacatcttcttcaaggagctgaactacaaaaccaattctgagtctccctctgtcacgatggtcaccctcctgtccaacctgggcagccagtggagcctgtggttcggctcctcggtgttgtctgtggtggagatggctgagctcgtctttgacctgctggtcatcatgttcctcatgctgctccgaaggttccgaagccgatactggtctccaggccgagggggcaggggtgctcaggaggtagcctccaccctggcatcctcccctccttcccacttctgcccccaccccatgtctctgtccttgtcccagccaggccctgctccctctccagccttgacagcccctccccctgcctatgccaccctgggcccccgcccatctccagggggctctgcaggggccagttcctccacctgtcctctgggggggccctgagagggaaggagaggtttctcacaccaaggcagatgctcctctggtgggagggtgctggccctggcaagattgaaggatgtgcagggcttcctctcagagccgcccaaactgccgttgatgtgtggaggggaagcaagatgggtaagggctcaggaagttgctccaagaacagtagctgatgaagctgcccagaagtgccttggctccagccctgtaccccttggtactgcctctgaacactctggtttccccacccaactgcggctaagtctctttttcccttggatcagccaagcgaaacttggagctttgacaaggaactttcctaagaaaccgctgataaccaggacaaaacacaaccaagggtacacgcaggcatgcacgggtttcctgcccagcgacggcttaagccagcccccgactggcctggccacactgctctccagtagcacagatgtctgctcctcctcttgaacttgggtgggaaaccccacccaaaagccccctttgttacttaggcaattccccttccctgactcccgagggctagggctagagcagacccgggtaagtaaaggcagacccagggctcctctagcctcatacccgtgccctcacagagccatgccccggcacctctgccctgtgtctttcatacctctacatgtctgcttgagatatttcctcagcctgaaagtttccccaaccatctgccagagaactcctatgcatcccttagaaccctgctcagacaccattacttttgtgaacgcttctgccacatcttgtcttccccaaaattgatcactccgccttctcctgggctcccgtagcacactataacatctgctggagtgttgctgttgcaccatactttcttgtacatttgtgtctcccttcccaactagactgtaagtgccttgcggtcagggactgaatcttgcccgtttatgtatgctccatgtctagcccatcatcctgcttggagcaagtaggcaggagctcaataaatgtttgttgcatgaaaaaaaaaaaaaaaaaa

SEQ ID NO: 18 - protein, 640 aa, human
MHVKKYLLKGLHRLQKGPGYTYKELLVWYCDNTNTHGPKRIICEGPKKKAMWFLLTLLFAALVCWQWGIFIRTYLSWEVSVSLSVGFKTMDFPAVTICNASPFKYSKIKHLLKDLDELMEAVLERILAPELSHANATRNLNFSIWNHTPLVLIDERNPHHPMVLDLFGDNHNGLTSSSASEKICNAHGCKMAMRLCSLNRTQCTFRNFTSATQALTEWYILQATNIFAQVPQQELVEMSYPGEQMILACLFGAEPCNYRNFTSIFYPHYGNCYIFNWGMTEKALPSANPGTEFGLKLILDIGQEDYVPFLASTGGVRLMLHEQRSYPFIRDEGIYAMSGTETSIGVLVDKLQRMGEPYSPCTVNGSEVPVQNFYSDYNTTYSIQACLRSCFQDHMIRNCNCGHYLYPLPRGEKYCNNRDFPDWAHCYSDLQMSVAQRETCIGMCKESCNDTQYKMTISMADWPSEASEDWIFHVLSQERDQSTNITLSRKGIVKLNIYFQEFNYRTIEESAANNIVWLLSNLGGQFGFWMGGSVLCLIEFGEIIIDFVWITIIKLVALAKSLRQRRAQASYAGPPPTVAELVEAHTNFGFQPDTAPRSPNTGPYPSEQALPIPGTPPPNYDSLRLQPLDVIESDSEGDAI

SEQ ID NO: 19 - DNA, 2564 bp, human
tcgccgggtgtcccagtgtcaccaacactcggccgccgccgccagcttggcgcgcaccgccgcctccgccaccgccgacagcgcgcatcctccgtgtccccgctccgccgcccgagcaggtgccactatgcacgtgaagaagtacctgctgaagggcctgcatcggctgcagaagggccccggctacacgtacaaggagctgctggtgtggtactgcgacaacaccaacacccacggccccaagcgcatcatctgtgaggggcccaagaagaaagccatgtggttcctgctcaccctgctcttcgccgccctcgtctgctggcagtggggcatcttcatcaggacctacttgagctgggaggtcagcgtctccctctccgtaggcttcaagaccatggacttccccgccgtcaccatctgcaatgctagccccttcaagtattccaaaatcaagcatttgctgaaggacctggatgagctgatggaagctgtcctggagagaatcctggctcctgagctaagccatgccaatgccaccaggaacctgaacttctccatctggaaccacacacccctggtccttattgatgaacggaacccccaccaccccatggtccttgatctctttggagacaaccacaatggcttaacaagcagctcagcatcagaaaagatctgtaatgcccacgggtgcaaaatggccatgagactatgtagcctcaacaggacccagtgtaccttccggaacttcaccagtgctacccaggcattgacagagtggtacatcctgcaggccaccaacatctttgcacaggtgccacagcaggagctagtagagatgagctaccccggcgagcagatgatcctggcctgcctattcggagctgagccctgcaactaccggaacttcacgtccatcttctaccctcactatggcaactgttacatcttcaactggggcatgacagagaaggcacttccttcggccaaccctggaactgaattcggcctgaagttgatcctggacataggccaggaagactacgtccccttccttgcgtccacgggcggggtcaggctgatgcttcacgagcagaggtcataccccttcatcagagatgagggcatctacgccatgtcggggacagagacgtccatcggggtactcgtggataagcttcagcgcatgggggagccctacagcccgtgcaccgtgaatggttctgaggtccccgtccaaaacttctacagtgactacaacacgacctactccatccaggcctgtcttcgctcctgcttccaagaccacatgatccgtaactgcaactgtggccactacctgtacccactgccccgtggggagaaatactgcaacaaccgggacttcccagactgggcccattgctactcagatctacagatgagcgtggcgcagagagagacctgcattggcatgtgcaaggagtcctgcaatgacacccagtacaagatgaccatctccatggctgactggccttctgaggcctccgaggactggattttccacgtcttgtctcaggagcgggaccaaagcaccaatatcaccctgagcaggaagggaattgtcaagctcaacatctacttccaagaatttaactatcgcaccattgaagaatcagcagccaataacatcgtctggctgctctcgaatctgggtggccagtttggcttctggatggggggctctgtgctgtgcctcatcgagtttggggagatcatcatcgactttgtgtggatcaccatcatcaagctggtggccttggccaagagcctacggcagcggcgagcccaagccagctacgctggcccaccgcccaccgtggccgagctggtggaggcccacaccaactttggcttccagcctgacacggccccccgcagccccaacactgggccctaccccagtgagcaggccctgcccatcccaggcaccccgccccccaactatgactccctgcgtctgcagccgctggacgtcatcgagtctgacagtgagggtgatgccatctaaccctgcccctgtccaccccgggtgggtgaaactcactgagcagccaagactgttgcccgaggactcactgtatggtgccctctccaaagggtcgggagggtagctctccaggccagagcttgtgtccttcaacagagaggccagcggcaactggtccgttactggccaagggctctgaagaatcaacggtgctggtacaggatacaggaataaattgtatcttcacctggttcctaccctcgtccctacctgtcctgatcctggtcctgaagacccctcggaacaccctctcctggtggcaggccacttccctcccagtgccagtctccatccaccccagagaggaacaggcgggtgggccatgtggttttctccttcctggccttggctggcctctggggcaggggtggtggagagatggaagggcatcaggtgtagggaccctgccaagtggcacctgatttactctagaaaataaaagtagaaaatactgagaaaaaaaaaaaaaaaaaaa

SEQ ID NO: 20 - protein, 649 aa, human
MAPGEKIKAKIKKNLPVTGPQAPTIKELMRWYCLNTNTHGCRRIVVSRGRLRRLLWIGFTLTAVALILWQCALLVFSFYTVSVSIKVHFRKLDFPAVTICNINPYKYSTVRHLLADLEQETREALKSLYGFPESRKRREAESWNSVSEGKQPRFSHRIPLLIFDQDEKGKARDFFTGRKRKVGGSIIHKASNVMHIESKQVVGFQLCSNDTSDCATYTFSSGINAIQEWYKLHYMNIMAQVPLEKKINMSYSAEELLVTCFFDGVSCDARNFTLFHHPMHGNCYTFNNRENETILSTSMGGSEYGLQVILYINEEEYNPFLVSSTGAKVIIHRQDEYPSVEDVGTEIETTMVTSIGMHLTESFKLSEPSSQCTEGGSDVPIRNIYNAAYSLQICLHSCFQTKMVEKCGCAQYSQPLPPAANYCNYQQHPNWMYCYYQLHRAFVQEELGCQSVCKEACRFKEWTLTTSLAQWPSVVSEKWLLPVLTWDQGRQVNKKLNKTDLAKLLIFYKDLNQRSIMESPANSIEMLLSNFGGQLGLWMSCSVVCVIEIIEVFFIDFFSIIARRQWQKAKEWWAWKQAPPCPEAPRSPQGQDNPALDIDDDLPTFNSALHLPPALGTQVPGTPPPKYNTLRLERAFSNQLTDTQMLDEL

SEQ ID NO: 21 - DNA, 3384 bp, human
atggcccccggggagaagatcaaagccaaaatcaagaagaatctgcccgtgacgggccctcaggcgccgaccattaaagagctgatgcggtggtactgcctcaacaccaacacccatggctgtcgccgcatcgtggtgtcccgcggccgtctgcgccgcctcctctggatcgggttcacactgactgccgtggccctcatcctctggcagtgcgccctcctcgtcttctccttctatactgtctcagtttccatcaaagtccacttccggaagctggattttcctgcagtcaccatctgcaacatcaacccctacaagtacagcaccgttcgccaccttctagctgacttggaacaggagaccagagaggccctgaagtccctgtatggctttccagagtcccggaagcgccgagaggcggagtcctggaactccgtctcagagggaaagcagcctagattctcccaccggattccgctgctgatctttgatcaggatgagaagggcaaggccagggacttcttcacagggaggaagcggaaagtcggcggtagcatcattcacaaggcttcaaatgtcatgcacatcgagtccaagcaagtggtgggattccaactgtgctcaaatgacacctccgactgtgccacctacaccttcagctcgggaatcaatgccattcaggagtggtataagctacactacatgaacatcatggcacaggtgcctctggagaagaaaatcaacatgagctattctgctgaggagctgctggtgacctgcttctttgatggagtgtcctgtgatgccaggaatttcacgcttttccaccacccgatgcatgggaattgctatactttcaacaacagagaaaatgagaccattctcagcacctccatggggggcagcgaatatgggctgcaagtcattttgtacataaacgaagaggaatacaacccattcctcgtgtcctccactggagctaaggtgatcatccatcggcaggatgagtatccctccgtcgaagatgtgggaacagagattgagacaacaatggtcacctctataggaatgcacctgacagagtccttcaagctgagtgagccctccagtcagtgcacggagggcgggagtgacgtgccaatcaggaacatctacaacgctgcctactcgctccagatctgccttcattcatgcttccagacaaagatggtggagaaatgtgggtgtgcccagtacagccagcctctacctcctgcagccaactactgcaactaccagcagcaccccaactggatgtattgttactaccaactgcatcgagcctttgtccaggaagagctgggctgccagtctgtgtgcaaggaagcctgccgctttaaagagtggacactaaccacaagcctggcacaatggccatctgtggtttcggagaagtggttgctgcctgttctcacttgggaccaaggccggcaagtaaacaaaaagctcaacaagacagacttggccaaactcttgatattctacaaagacctgaaccagagatccatcatggagagcccagccaacagtattgagatgcttctgtccaacttcggcggtcagctgggcctgtggatgagctgctctgttgtctgcgtcatcgagatcatcgaggtcttcttcattgacttcttctctatcattgcccgccgccagtggcagaaagccaaggagtggtgggcctggaaacaggctcccccatgtccagaagctccccgtagcccacagggccaggacaatccagccctggatatagacgatgacctacccactttcaactctgctttgcacctgcctccagccctaggaacccaagtgcccggcacaccgccccccaaatacaataccttgcgcttggagagggccttttccaaccagctcacagatacccagatgctggatgagctctgaggcagggttgagaagacagatctagtcaggaccaccagccatggtctaaggacatggatcgggtgcccccagacgtgtgcacaggggaccctctgccccactctgggcttttcagatactctgaccaaaaagcctgctttaaaccgcaagatggggcctgggcatgcgcaggaggagccatcgggtactacgcagcaacactcacaactgtccaggctgagataaatcccgggacctgaactattagcacgtcactagagactgggagccgaggcagtggtgctggcccaagtgaaggccagagtgaggactgatgcagctctttacgggtcttgagagggaaggactcttccaaagccccaaagccgagggtttcacccacactgccagcctgggttggggcccaaggatgtgaccttgagtgtcaaggctggacagctactgccagatgccaaagataggagaaagtgccagccctgaagctggagccgcttgtgaataaactgttcttcatcattgacactggagaaaggtgtcctccatgccctcaggcagcagagaactggcccagagcccttggagtgttggtggagatcagagtgccgtggtggaggtctgggactatgtcagagtgtcctcactttggggcatgggtggctccaggagatggatttagttattcaattttgtggatgaataaattgaggcacagaaagattaagttaccggcccaaggtgacacagtgaggaggtggcagagctaggatttgaccccagacaatctgacttcatgattgtggcatccaattcgtgtctgtgcctcaatttgtgcaaacgactgtgtcactttgcttggggagggggagcatcccacccacatgccctgggcaggtgacccaagacagagctgacccctccaccaccaaaggcctgtctctcaccaacaagccaactgctcacagatgacccacttcataccacattcacatctggccacctgactccagctatcaggtattaagtcccgggcagcgataagcccccccacaacagtgtgttagctctgtcttagcaacctgataggttagcataggggctgcaaatgttgttccgccacaccaaatccctgtttttgtccatgaactaagaattttattttattttattttttattttttgtagaaataggatctcactatgttgcccaggctgttcttgaactcctggcctcaaatgatcttcccacttcagcctcccaaagtgcagggattacaggcacaagccaccgtgcccagccaagaatttttatttttgcttttaaatacccaaagaagaagattctgtgactcatggaaatgatatgagattcgaattccagtgtctataaataaagttttattggtacacagccccaaaaaaaaaaaaaaaaaaaaa

SEQ ID NO: 22 - protein, 638 aa, human
MAEHRSMDGRMEAATRGGSHLQAAAQTPPRPGPPSAPPPPPKEGHQEGLVELPASFRELLTFFCTNATIHGAIRLVCSRGNRLKTTSWGLLSLGALVALCWQLGLLFERHWHRPVLMAVSVHSERKLLPLVTLCDGNPRRPSPVLRHLELLDEFARENIDSLYNVNLSKGRAALSATVPRHEPPFHLDREIRLQRLSHSGSRVRVGFRLCNSTGGDCFYRGYTSGVAAVQDWYHFHYVDILALLPAAWEDSHGSQDGHFVLSCSYDGLDCQARQFRTFHHPTYGSCYTVDGVWTAQRPGITHGVGLVLRVEQQPHLPLLSTLAGIRVMVHGRNHTPFLGHHSFSVRPGTEATISIREDEVHRLGSPYGHCTAGGEGVEVELLHNTSYTRQACLVSCFQQLMVETCSCGYYLHPLPAGAEYCSSARHPAWGHCFYRLYQDLETHRLPCTSRCPRPCRESAFKLSTGTSRWPSAKSAGWTLATLGEQGLPHQSHRQRSSLAKINIVYQELNYRSVEEAPVYSVPQLLSAMGSLCSLWFGASVLSLLELLELLLDASALTLVLGGRRLRRAWFSWPRASPASGASSIKPEASQMPPPAGGTSDDPEPSGPHLPRVMLPGVLAGVSAEESWAGPQPLETLDT

SEQ ID NO: 23 - DNA, 2503 bp, human
actcgggaaggccacacagccagtgacgaagctgtgattcacacaggcctgggtgactccagcatggctttcctctccaggacgtcaccggtggcagctgcttccttccagagccggcaggaggccagaggctccatcctgcttcagagctgccagctgcccccgcaatggctgagcaccgaagcatggacgggagaatggaagcagccacacgggggggctctcacctccaggctgcagcccagacgccccccaggccggggccaccatcagcaccaccaccaccacccaaggaggggcaccaggaggggctggtggagctgcccgcctcgttccgggagctgctcaccttcttctgcaccaatgccaccatccacggcgccatccgcctggtctgctcccgcgggaaccgcctcaagacgacgtcctgggggctgctgtccctgggagccctggtcgcgctctgctggcagctggggctcctctttgagcgtcactggcaccgcccggtcctcatggccgtctctgtgcactcggagcgcaagctgctcccgctggtcaccctgtgtgacgggaacccacgtcggccgagtccggtcctccgccatctggagctgctggacgagtttgccagggagaacattgactccctgtacaacgtcaacctcagcaaaggcagagccgccctctccgccactgtcccccgccacgagccccccttccacctggaccgggagatccgtctgcagaggctgagccactcgggcagccgggtcagagtggggttcagactgtgcaacagcacgggcggcgactgcttttaccgaggctacacgtcaggcgtggcggctgtccaggactggtaccacttccactatgtggatatcctggccctgctgcccgcggcatgggaggacagccacgggagccaggacggccacttcgtcctctcctgcagttacgatggcctggactgccaggcccgacagttccggaccttccaccaccccacctacggcagctgctacacggtcgatggcgtctggacagctcagcgccccggcatcacccacggagtcggcctggtcctcagggttgagcagcagcctcacctccctctgctgtccacgctggccggcatcagggtcatggttcacggccgtaaccacacgcccttcctggggcaccacagcttcagcgtccggccagggacggaggccaccatcagcatccgagaggacgaggtgcaccggctcgggagcccctacggccactgcaccgccggcggggaaggcgtggaggtggagctgctacacaacacctcctacaccaggcaggcctgcctggtgtcctgcttccagcagctgatggtggagacctgctcctgtggctactacctccaccctctgccggcgggggctgagtactgcagctctgcccggcaccctgcctggggacactgcttctaccgcctctaccaggacctggagacccaccggctcccctgtacctcccgctgccccaggccctgcagggagtctgcattcaagctctccactgggacctccaggtggccttccgccaagtcagctggatggactctggccacgctaggtgaacaggggctgccgcatcagagccacagacagaggagcagcctggccaaaatcaacatcgtctaccaggagctcaactaccgctcagtggaggaggcgcccgtgtactcggtgccgcagctgctctcggccatgggcagcctctgcagcctgtggtttggggcctccgtcctctccctcctggagctcctggagctgctgctcgatgcttctgccctcaccctggtgctaggcggccgccggctccgcagggcgtggttctcctggcccagagccagccctgcctcaggggcgtccagcatcaagccagaggccagtcagatgcccccgcctgcaggcggcacgtcagatgacccggagcccagcgggcctcatctcccacgggtgatgcttccaggggttctggcgggagtctcagccgaagagagctgggctgggccccagccccttgagactctggacacctgaaccagacctgccagggctgtgcgatctcttggcctggtccttgcagctgtggcagcagcaggctccccagcggcccagggtgggccagaccagcagcccaggaagcagcacacgcggccgtggggaggcaggcaccgggcatgtcggcgcctctggtcaaaccacctacactgcctggggtgggtctcaaggaggcccggggcggaggggggttcccgcgtgcacacgagtgcggctggacgtgccgacacgcggtgatgtacccatgctccgtgtgtctgtgtctgcatgtccacacgtctgatgcacctgtgtacgtgtgtcaagcctagccacctcagctgcagggaggcagaaggcaaggcaggccccacggacacacttgggctgctctgaaataaagctgttgactccacctg

(19) United States
(12) Patent Application Publication
(10) Pub. No.: US 2009/0075320 A1
(43) Pub. Date: Mar. 19, 2009

(54) Method for screening an enhancer or a masker of salty taste

(76) Inventors: Jannetje Wilhelmina Bos, Vlaardingen (NL); Robertus Johannes Gouka, AT Vlaardingen (NL); Erwin Werner Tareilus, AT Vlaardingen (NL)

Correspondence Address: MORGAN LEWIS & BOCKIUS LLP, 1111 PENNSYLVANIA AVENUE NW, WASHINGTON, DC, 20004, US

(21) Appl. No.: 11/878,098

(22) Filed: Jul. 20, 2007

(30) Foreign Application Priority Data
Jul. 21, 2006 (EP) EP06117694

(51) Int. Cl. C12Q 1/68; C07H 21/00; C12N 15/63; C12N 5/00

(52) U.S. Cl. 435/29; 536/23.1; 435/320.1; 435/325

(57) ABSTRACT

The invention relates to a nucleic acid molecule being represented by a nucleic acid sequence comprising at least three nucleic acid subsequences, each nucleic acid subsequence coding for one of the α, β, and γ subunits of the epithelial sodium channel, and each nucleic acid subsequence being arranged in a head to tail configuration with respect to another. The invention further relates to a cell comprising the nucleic acid sequence of the invention, which is able to express the subunits and to its use in a method for screening a potential modulator compound of the subunits of the epithelial sodium channel.

### FIELD OF THE INVENTION

The invention relates to an expression system for the subunits of the epithelial sodium channel, to a cell expressing these subunits and to the use of these cells in a method for screening a potential modulator compound of the epithelial sodium channel.

### BACKGROUND OF THE INVENTION

The coding DNA (cDNA) for the α, β and γ subunits of the human epithelial sodium channel (ENaC) have already been isolated and their genes have been cloned (Voilley et al, (1994), Proc. Natl. Acad. Sci., 91:247-251 and Voilley et al, (1995), Genomics, 28:560-565). Each cDNA codes for one transmembrane protein having a large extracellular domain. All subunits are thought to be required for formation of a functional sodium channel (Canessa et al, (1994), Nature, 367:463-467). Several models have been proposed as to the stoichiometry of the α, β and γ subunits in order to form a functional channel (Kosari et al, (1998), J. Biol. Chem., 273:13469-13474 and Staruschenko et al, (2005), Biophys. J., 88:3966-3975). ENaC is implicated in a broad spectrum of cellular functions by controlling fluid and electrolyte transport across epithelia in many organs such as kidney, colon, lung (Palmer et al, (1986), Proc. Natl. Acad. Sci., 83: 2767-2770 and Voilley et al, (1994), Proc. Natl. Acad. Sci., 91:247-251). Furthermore, in the taste buds of the tongue these ENaC are thought to be the starting points for salt taste transduction towards the cerebrumn of the brain (Heck et al, (1984), Science, 223:403-405). Whereas the evidence for a central role of ENaC in salt taste transduction of rodents is very strong, only limited information is available for the salt detection mechanisms in humans. Several ENaC family members have been found in humans. Taste perception in humans is suspected to be mediated by a combination of several ENaC family members. The ENaC defined above is highly selective for sodium, lithium and protons. Several screening systems have already been developed to try to identify modulators of ENaC. Such modulators could be either enhancers or maskers of salt perception. Enhancers of salt perception could be used for example in food. It will allow decreasing the salt content of food, while retaining an acceptable salt taste. On the other hand, maskers of salt perception could be used to mask undesirable salt perception. Alternatively, maskers could be used to reduce salt, preferably sodium intake in order to decrease blood pressure. The screening systems developed so far are usually in vivo based assays. ENaC subunits are expressed in a given cell system and the effect of the addition of a potential modulator on the ENaC is monitored in the presence of a cation. Data for a transient expression of the ENaC subunits in a mammalian cell line HEK293 was described in US 2004/0072254 A1. However, in our experience, mammalian cells stably expressing the ENaC subunits resulted in cells with strongly reduced vitality. It seems as mammalian cells appear to be unable to cope with massive and continuous sodium influx over long time periods. Therefore, a screening assay based on a stable expression system cannot be easily achieved in mammalian cells. The alternative of transient expression was also documented in the same patent application. However, it introduces large experimental error by variable expression levels. Therefore, this alternative does not seem suitable for high throughput screening. Therefore, there is still a need for an improved ENaC expression systems that can be used in an efficient screening assay to identify ENaC modulators.

### DESCRIPTION OF THE FIGURES

FIG. 1. This figure shows the vector used in the expression system of the invention. This vector is based on pcDNA5/FRT/TO and contains the nucleic acid subsequences for three subunits of the human epithelial sodium channel arranged in a head to tail orientation (alpha, beta, gamma). Each of them was preceded with a tetracycline-inducible promoter. The plasmid contained an FRT (Flp Recombination Target) site, which allows the site-specific integration of the plasmid DNA via Flp recombinase-mediated DNA recombination at the FRT site of a mammalian cell line having a FRT site in his genome.

FIG. 2. Western blot with samples of HEK293 Flp-In T-Rex hENaC cells (using equivalent amount of proteins per lane): The lanes contain protein samples from HEK293 Flp-In T-Rex cells that were either untransfected or transfected with pUR8191 or, as for the anti-V5 antibody, with a vector containing a γ-V5 derivative, obtained from induced (+doxycyclin) or non-induced (−doxycyclin) conditions. The immunoblots were developed with anti-α, anti-β and anti-V5 tag antibodies.

FIG. 3. MP-Blue signal in HEK293-hENaC cells (pUR8191) after 24 h+/−induction. After 30 seconds, cells were challenged by addition of 140 mM NaCl- or NMDG-containing buffer, resulting in a final concentration of 100 or 0 mM extracellular Na⁺. The fluorescence signals were normalised at t=0, averages were determined for 12 wells.

FIG. 4. Dose-response curve for different concentrations of the hENaC blockers amiloride, benzamil and phenamil (in M). Cells were loaded with MP-Blue dye for 1 hr and assayed in the Flexstation.

FIG. 5. HEK293-hENaC cells were seeded in 96-wells, grown for 24 hrs, induced, loaded with SBFI for 1 hr and assayed in the Flexstation with a filter combination of 340/510 and 380/510 nm. After 30 seconds, cells were challenged by addition of 140 mM NaCl, resulting in a final concentration of 100 mM extracellular Na⁺. Average was determined for 12 wells.

FIG. 6 (not shown). This figure is similar to FIG. 1. It shows the vector used in the expression system of the invention. This vector is based on pcDNA5/FRT/TO and contains the nucleic acid subsequences for three subunits of the human epithelial sodium channel arranged in a head to tail orientation (delta, beta, gamma). Each of them was preceded with a tetracycline-inducible promoter. The plasmid contained an FRT (Flp Recombination Target) site, which allows the site-specific integration of the plasmid DNA via Flp recombinase-mediated DNA recombination at the FRT site of a mammalian cell line having a FRT site in his genome. The vector is named pUR8245.

FIG. 7 (not shown). This figure is also similar to FIG. 1. It shows the vector used in the expression system of the invention. This vector is based on pcDNA5/FRT/TO and contains the nucleic acid subsequences for four subunits of the human epithelial sodium channel arranged in a head to tail orientation (alpha, beta, gamma, delta). Each of them was preceded with a tetracycline-inducible promoter. The plasmid contained an FRT (Flp Recombination Target) site, which allows the site-specific integration of the plasmid DNA via Flp recombinase-mediated DNA recombination at the FRT site of a mammalian cell line having a FRT site in his genome. The vector is named pUR2846.

FIG. 8 (not shown). This figure is similar to FIG. 3. It shows 3 MP-Blue signal in HEK293-hENaC cells (pUR8245) after 24 h+/−induction. After 30 seconds, cells were challenged by addition of 140 mM NaCl- or NMDG-containing buffer, resulting in a final concentration of 100 or 0 mM extracellular Na⁺. The fluorescence signals were normalised at t=0.

FIG. 9 (not shown). This figure is similar to FIG. 3. It shows MP-Blue signal in HEK293-hENaC cells (pUR8246) after 24 h+/−induction. After 30 seconds, cells were challenged by addition of 140 mM NaCl- or NMDG-containing buffer, resulting in a final concentration of 100 or 0 mM extracellular Na⁺. The fluorescence signals were normalised at t=0.

FIG. 10 (not shown). This figure is similar to FIG. 5. HEK293-hENaC cells (pUR8245) were seeded in 96-wells, grown for 24 hrs, induced, loaded with SBFI for 1 hr and assayed in the Flexstation with a filter combination of 320/510 and 380/510 nm. After 30 seconds, cells were challenged by addition of 140 mM NaCl, resulting in a final concentration of 100 mM extracellular Na⁺. Average was determined for 6 wells.

FIG. 11 (not shown). This figure is similar to FIG. 5. HEK293-hENaC cells (pUR8246) were seeded in 96-wells, grown for 24 hrs, induced, loaded with SBFI for 1 hr and assayed in the Flexstation with a filter combination of 320/510 and 380/510 nm. After 30 seconds, cells were challenged by addition of 140 mM NaCl, resulting in a final concentration of 100 mM extracellular Na⁺. Average was determined for 6 wells.

### DESCRIPTION OF THE INVENTION

### Nucleic Acid Molecule

In a first aspect, the invention provides a nucleic acid molecule being represented by a nucleic acid sequence comprising at least three nucleic acid subsequences arranged in a head to tail configuration with respect to another. Two of these nucleic acid subsequences code for the β and γ subunits of the epithelial sodium channel respectively or a variant, fragment or functional equivalent of each of these subunits. Additional nucleic acid subsequence(s) is(are) selected from the group consisting of: a nucleic acid subsequence coding for the α subunit and a nucleic acid subsequence coding for the δ subunit of the epithelial sodium channel or a variant, fragment or functional equivalent of each of these subunits.

A nucleic acid subsequence is herein defined as a nucleic acid sequence encoding a given polypeptide. A nucleic acid subsequence can be a coding sequence (cDNA) or a genomic DNA sequence. Each nucleic acid subsequence present in the nucleic acid sequence of the invention is defined herein as a nucleic acid sequence, which when transcribed into mRNA will be subsequently translated into one of the subunits of the epithelial sodium channel or ENaC as defined above.

The following combinations of subunits of the ENaC are therefore preferably present in the nucleic acid molecule of the invention:
the α, β and γ subunits of the epithelial sodium channel respectively or a variant, fragment or functional equivalent of each of these subunits,
the δ, β, and γ subunits of the epithelial sodium channel respectively or a variant, fragment or functional equivalent of each of these subunits,
the α, δ, β and γ subunits of the epithelial sodium channel respectively or a variant, fragment or functional equivalent of each of these subunits,

These three combinations of subunits of the ENaC are expected to be functional when expressed in a suitable host cell as exemplified later on in the description of the invention. Each combination of subunits as defined above is expected to represent a functional epithelial sodium channel or ENaC.

The functionality of the combination of subunits of the ENaC is preferably assessed when expressed in a suitable host cell by measuring cation-, preferably sodium-mediated changes in signal of the cell. This assessment is preferably performed as described in steps a) to d) of the method of the invention later described herein except that no potential modulator of the subunits is added. The cation-mediated changes in signal of the cell is an indicator of the subunits (or channel) functionality.

The nucleic acid subsequence encoding ENaC subunits employed can be naturally occurring forms, variants containing SNPs (Single Nucleotide Polymorphisms), alternatively spliced forms, combinations of forms, or any functional variants known in the art.

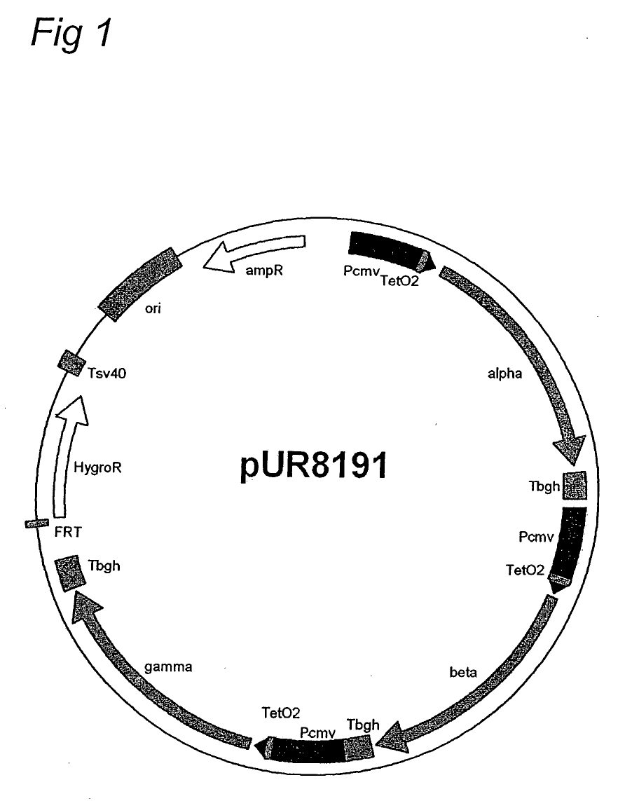

Several nucleic acid subsequences coding for the above-defined subunits have already been disclosed. The coding DNA (cDNA) for the α, β and γ subunits of the human epithelial sodium channel (ENaC) were first disclosed in two publications of Voilley (Voilley et al, (1994), Proc. Natl. Acad. Sci., 91:247-251 and Voilley et al, (1995), Genomics, 28:560-565). Other publications later disclosed similar human coding DNA for these subunits: UK 2 396 414 A (confere FIG. 1a for α, FIG. 1b for β, and FIG. 1c for γ) or accession number NM_{—}002978 (for δ in NCBI database). US 2004/0072254 A1 also discloses human cDNA sequences for the α, β, γ, and δ subunits as SEQ ID NO:1, 2, 3, and 7. Alternatively, spliced forms of the rat alpha chain have already been disclosed in U.S. Pat. No. 5,693,756.

According to a preferred embodiment, each nucleic acid subsequence encoding a subunit of the ENaC as defined above is selected from the group consisting of:
1) each encoded subunit of the ENaC has an amino acid sequence that has greater than about 80% amino acid sequence identity to an amino acid sequence encoded by the corresponding nucleic acid subsequence having the following SEQ ID NO:
   SEQ ID NO:2, and/or SEQ ID NO:19 for the β subunit, and
   SEQ ID NO:3, and/or SEQ ID NO:21 for the γ subunit, and
   SEQ ID NO:1, and/or SEQ ID NO:17 or for the α subunit, and/or
   SEQ ID NO:7, and/or SEQ ID NO:23 for the δ subunit, and/or
2) the α subunit of the ENaC has an amino acid sequence has greater than about 80% amino acid sequence identity to the amino acid sequence SEQ ID NO:15 and/or comprises the amino acid fragment as given in SEQ ID NO:12 or SEQ ID NO:13 or SEQ ID NO:14, and/or
3) each encoded subunit of the ENaC specifically binds to antibodies, e.g. polyclonal antibodies, raised against an immunogen comprising an amino acid sequence encoded by one of the corresponding nucleic acid subsequences as defined in 1) or 2), or immunogenic fragments thereof, and conservatively variants thereof, and/or
4) each nucleic acid subsequence encoding the subunit specifically hybridizes under stringent conditions to the corresponding nucleic acid subsequences as defined in 1) or 2) or their complements, and conservatively modified variants thereof.

The nucleic acid subsequences having SEQ ID NO:1 or SEQ ID NO:17 codes for the α chain subunit having an amino acid sequence corresponding to SEQ ID NO:4 and SEQ ID NO:16 respectively.

The nucleic acid subsequences having SEQ ID NO:2 or SEQ ID NO:19 codes for the β chain subunit having an amino acid sequence corresponding to SEQ ID NO:5 and SEQ ID NO:18 respectively.

The nucleic acid subsequences having SEQ ID NO:3 or SEQ ID NO:21 codes for the γ chain subunit having an amino acid sequence corresponding to SEQ ID NO:6 and SEQ ID NO:20 respectively.

The nucleic acid subsequences having SEQ ID NO:7 or SEQ ID NO:23 codes for the δ chain subunit having an amino acid sequence corresponding to SEQ ID NO:8 and SEQ ID NO:22 respectively.

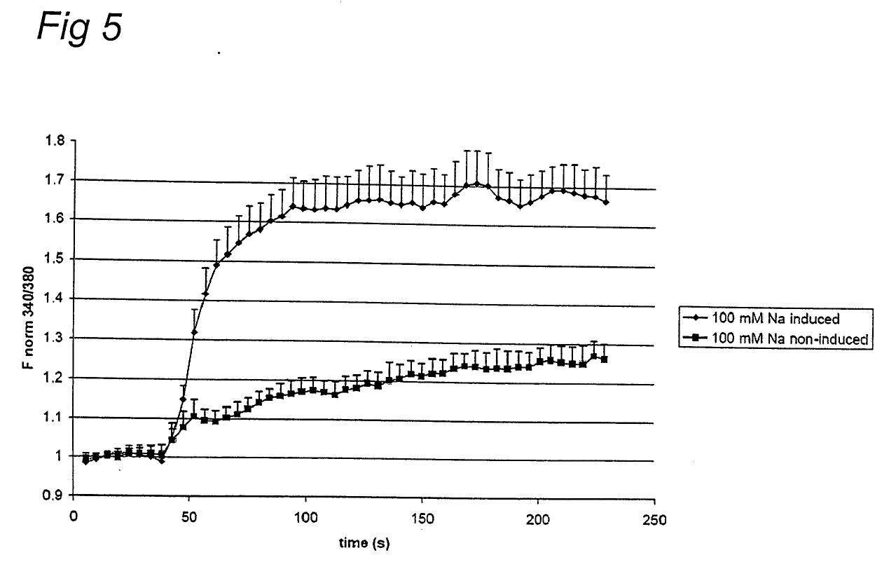

Each amino acid fragment SEQ ID NO:12 or SEQ ID NO:13 or SEQ ID NO:14 is encoded by the following nucleic acid sequences SEQ ID NO:9, SEQ ID NO:10, SEQ ID NO:11 respectively. Each of these amino acid fragment starts at position Glu 470 of the corresponding whole amino acid subsequence (as indicated on FIG. 5B of U.S. Pat. No. 5,693,756). The amino acid fragment SEQ ID NO:12 is part of the amino acid subsequence SEQ ID NO:15. The amino acid subsequence SEQ ID NO:15 is the rat α subunit of the ENaC. The two α subunits defined in 2) comprising amino acid fragment SEQ ID NO:13 or SEQ ID NO:14 represent spliced forms of the rat alpha chain as disclosed in U.S. Pat. No. 5,693,756.

In a more preferred embodiment, the nucleic acid molecule of the invention is represented by a nucleic acid sequence comprising at least three nucleic acid subsequences arranged in a head to tail configuration with respect to another, wherein two of these nucleic acid subsequences code for the β and γ subunits of the epithelial sodium channel, and additional nucleic acid subsequence(s) is(are) selected from the group consisting of: a nucleic acid subsequence coding for the α subunit and a nucleic acid subsequence coding for the δ subunit of the epithelial sodium channel, and wherein each encoded subunit of the epithelial sodium channel as identified above has an amino acid sequence that has about 85% or more amino acid sequence identity with the amino acid sequence encoded by the corresponding nucleic acid subsequence having the following SEQ ID NO:
SEQ ID NO:19 for the β subunit, and
SEQ ID NO:21 for the γ subunit, and
SEQ ID NO:17 for the α subunit, and/or
SEQ ID NO:23 for the δ subunit respectively.

This preferred combination of subunit concerns subunits, which do not originate from a rat. More preferably, they originate from a human being. Even more preferably, the nucleic acid molecule as defined in this preferred embodiment is for functional expression in mammalian, even more preferably human cells. The use of sequences, which are highly homologous (identity of about or more than 85%) with human sequences is attractive since we may expect these nucleic acid molecule will be expressed and functional in mammalian, preferably human cells. Furthermore, these sequences are so highly homologous with human sequences that we expect that the cell type hence prepared will mimic human taste more efficiently than cell type prepared with rat sequences for example. However, a stable and functional high-level expression of sequences which are highly homologous with human sequences has been so far very difficult to obtain. This is the reason for the use of less preferred expression systems by others thus far: either transient expression systems (WO 2002/087306 A2) or use of cell lines endogenously expressing animal homologues of the human channel subunits (WO 2004/072645 A2).

Even more preferably, the identity as defined earlier herein is about 85% or more, even more preferably about 90% or more, even more preferably about 91% or more, even more preferably about 92% or more, even more preferably about 93% or more, even more preferably about 94% or more, even more preferably about 95% or more, even more preferably about 96% or more, even more preferably about 97% or more, even more preferably about 98% or more, even more preferably about 99% or more, and most preferably about 100%.

Percentage of identity was determined by calculating the ratio of the number of identical nucleotides in the sequence divided by the length of the total nucleotides minus the lengths of any gaps. DNA multiple sequence alignment was performed using a DNA alignment programme such as DNAman version 4.0, using the Optimal Alignment (Full Alignment) program, or the Align Plus software of Clone Manager Suitc.

Stringent hybridization conditions mean prehybridization and hybridization at 42° C. in 5×SSPE, 0.3% SDS, 200 pg/ml sheared and denatured salmon sperm DNA, and 50% formamide. Subsequently, the hybridization reaction is washed three times for 30 minutes using 2×SSC, 0.2% SDS at 65° C.

Preferably the nucleic acid subsequences used are from human origin. Even more preferably, the nucleic acid subsequences coding for the above-defined subunits are the following ones:
the α chain subunit used has the following amino acid sequence SEQ ID NO:4 or SEQ ID NO:16, which is encoded by the following nucleic acid subsequence SEQ ID NO:1 or SEQ ID NO:17 respectively, and/or
the α chain subunit has the following amino acid sequence SEQ ID NO:15 and/or comprises either the amino acid fragment having SEQ ID NO:12, or SEQ ID NO:13 or SEQ ID NO:14 and/or
the β chain subunit used has the following amino acid sequence SEQ ID NO:5 or SEQ ID NO:18, which is encoded by the following nucleic acid subsequence SEQ ID NO:2 or SEQ ID NO:19 respectively, and/or
the γ chain subunit used has the following amino acid sequence SEQ ID NO:6 or SEQ ID NO:20, which is encoded by the following nucleic acid subsequence SEQ ID NO:3 or SEQ ID NO:21 respectively, and/or
the δ chain subunit used has the following amino acid sequence SEQ ID NO:8 or SEQ ID NO:22, which is encoded by the following nucleic acid subsequence SEQ ID NO:7 or SEQ ID NO:23 respectively,

Alternatively, and according to another preferred embodiment, the nucleic acid subsequences used are variants of nucleic acid subsequences as earlier defined herein. A variant of a nucleic acid subsequence as defined herein is preferably functional when the expression of a corresponding introduced nucleic acid molecule as defined herein is functional in the assay as earlier defined herein (steps a) top d) of the method of the invention without potential modulator of the subunit). Variants of nucleic acid sequences may be a fragment of these nucleic acid subsequences. A preferred variant contains silent mutations. Alternatively, or in combination, a nucleic acid sequence variant may also be obtained by introduction of nucleotide substitutions which do not give rise to another amino acid sequence, but which corresponds to the codon usage of the host cell wherein the nucleic acid subsequences will be expressed. Preferably, the nucleic acid subsequence variant is such that starting from any one of the nucleic acid subsequences as earlier defined herein one or more nucleotides from the 5′ and/or 3′ end have been deleted. Alternatively or in combination, a nucleic acid subsequence variant is preferably a nucleic acid sequence isolated from other organisms and/or another family member of the nucleic acid subsequences as earlier defined herein. All these variants can be obtained in a typical approach, using cDNA or genomic libraries from a chosen species. The library can be subsequently screened with one of the nucleic acid subsequences as earlier defined herein or part thereof by hybridization under stringent conditions as defined above. Human is a preferred species. According to another preferred embodiment, a nucleic acid subsequence variant is an allelic variant. An allelic variant denotes any of two or more alternative forms of a gene occupying the same chromosome locus. Allelic variation arises naturally through mutation, and may result in phenotypic polymorphism within populations.

According to another preferred embodiment, a nucleic acid subsequence variant differs from any of the nucleic acid subsequences as earlier defined herein by virtue of the degeneracy of the genetic code.

A head to tail configuration is herein defined as a configuration in which the direction of transcription of each nucleic acid subsequence is similar to each other to form a single nucleic acid molecule. This has been exemplified in Rao et al (Rao et al, (2000), Analytical Biochemistry, 286:206-213). This single nucleic acid molecule can be subsequently introduced into a cell. Using a head to tail configuration is advantageous for the present invention, since the expression system obtained is expected to be more reproducible and more stable than previous systems. Furthermore, using a single nucleotide acid molecule comprising at least three of the subunits in a head to tail arrangement is expected to control the co-expression level of the at least three subunits in such a way that the physiological/functional stoichiometry of ENaC may be respected.

Even more preferably, the expression of each nucleic acid subsequence present in the nucleic acid sequence of the invention defined above is inducible. The inducibility of the expression of each nucleic acid subsequence can be fulfilled by any ways known to the skilled person. For example, the Invitrogen T-Rex system (Tetracycline-Regulated Expression, based on the tet operon, expression of the inserted gene is repressed until an inducer is added to the media), the Invitrogen Gene-Switch System (based on activation of the Gal4-E1b promoter), the Stratagene Complete Control Inducible mammalian Expression system (based on transcription activation by the insect hormone ecdysone or its analog ponasterone A (ponA) in mammalian cells harboring both the gene for the Drosophila melanogaster ecdysone receptor and a promoter containing a binding site for the ecdysone receptor), the New England Biolabs RheoSwitch® Mammalian Inducible Expression System (based on the highly specific interaction of a synthetic inducer, RheoSwitch Ligand RSL1, and a chimeric bipartite nuclear receptor), the Qbiogene Q-mate™ Inducible Expression System, or Q-mate™ CymR system (based on repression of gene expression by the cumate repressor protein CymR bound to operator sites in the absence of the inducer molecule cumate. With cumate present, CymR binds to cumate and undergoes a conformational change resulting in its release from the operator sites.), the Stratagene's LacSwitch® II inducible mammalian expression system (based on the lac operon, expression of the inserted gene is repressed until an inducer is added to the media).

Even more preferably, the expression of each nucleic acid subsequence is rendered inducible by the presence of an inducible promoter operably linked to each of the nucleic acid subsequence present in the nucleic acid sequence of the invention.

In the context of the invention, “operably linked” is defined as a configuration in which a control sequence, here a promoter sequence, is appropriately placed at a position relative to the nucleic acid subsequence such that the control sequence directs the expression of the nucleic acid subsequence.

An inducible promoter may be any promoter or parts thereof functional for a given nucleic acid subsequence as defined above and in a given cell, wherein the transcription initiation activity of the promoter can be induced in a cell upon the addition of a given inducing agent during culture of the cells.

More preferably, the inducible promoter is a tetracycline-regulated promoter. Even more preferably, the inducible promoter is a tetracyclin-regulated hybrid human cytomegalovirus promoter as described in Yao et al (Yao, F. et al. (1998) Hum. Gene Therapy 9: 1939-1950 and Yao, F. and Eriksson, E. (1999) Hum. Gene Therapy 10: 419-427) This system can be purchased from Invitrogen as the T-Rex expression system).

The use of an inducible expression system may circumvent the toxicity problem described using stable expression systems.

Therefore the use of an inducible expression system with a head to tail configuration has several advantages compared to classical expression systems used so far in a screening method for identifying modulators of ENaC.

### Nucleic Acid Construct and Expression Vectors

In a second aspect, the invention provides a nucleic acid construct comprising the nucleic acid molecule of the invention being represented by a nucleic acid sequence of the invention as defined above.

Nucleic acid construct is herein defined as a nucleic acid molecule, either single-or-double stranded, which is isolated from a naturally occurring gene or which has been modified to contain segments of nucleic acid which are combined or juxtaposed in a manner which would not otherwise exist in nature. The term nucleic acid construct is synonymous with the term expression cassette when the nucleic acid construct contains all the control sequences required for expression of a coding sequence. The boundaries of the coding sequence are generally determined by the ATG start codon at the 5′end of the mRNA and a translation stop codon sequence terminating the open reading frame at the 3′end of the mRNA. Alternatively, a coding sequence can include, but is limited to, nucleic acid sequence (DNA), cDNA and recombinant nucleic acid sequences. Preferably, each nucleic acid subsequence present in the nucleic acid sequence of the invention is operably linked to one or more additional control sequences, which direct the production of each nucleic acid subsequence, that is to say of each subunit in a suitable expression host.

The term “control sequences” is defined herein to include all components, which are necessary or advantageous for expression of the subunits. Each control sequence may be native or foreign for the nucleic acid subsequence encoding the subunit. Such control sequences include, but are not limited to, a leader, optimal translation initiation sequences, a polyadenylation sequence, a promoter and a transcription terminator. At a minimum, the control sequences include a promoter, and transcriptional and translational stop signals. The control sequences may be provided with linkers for the purpose of introducing specific restriction sites facilitating ligation of the control sequences with the coding region of the gene encoding the subunit.

In a further aspect, there is provided an expression vector comprising the nucleic acid construct as defined above and transcriptional and translational stop signals.

A preferred promoter is the human cytomegalovirus immediate-early (CMV) promoter, especially when the host cell is human and/or when the nucleic acid subsequence is of human origin.

### Host Cell

In a further aspect, there is provided a host cell comprising the nucleic acid construct or the expression vector both as defined earlier. The skilled person will know that the choice of the cell depends largely on the origin of the nucleic acid subsequence encoding the subunit chosen. Any cell can be chosen as long as the subunits as expressed are functional. Preferably, the expression of the subunits is stable, optionally inducible. Furthermore, a functional channel is formed as earlier defined. Preferably, the cell is a prokaryote or an eukaryote cell. More preferably, the cell is an insect or a mammalian cell. Even more preferably, the mammalian cell is a human cell. Examples of mammalian cells are HEK293, HEK293T, MDCK, CHO, COS, NIH3T3, Swiss3T3, BHK, and A549. Even more preferably, the cell is a mammalian cell such as HEK293. The cell of the invention may be seen as a recombinant cell. The cell of the invention is advantageously used in the method of the invention as presented below. Three types of host cells may be prepared:
expressing α, β and γ subunits or
expressing β, γ and δ subunits or
expressing α, β, γ and δ subunits all as earlier defined herein.

### Method

In a further aspect, the invention relates to an in vitro method for screening a potential modulator compound of the subunits of the epithelial sodium (or ENaC) channel using the cell of the invention previously defined. This method is not practiced on human body.

Potential modulators of the ENaC channel are herein defined as compounds that can block, inhibit, modulate or enhance salty taste perception by blocking, inhibiting, modulating or enhancing the ENaC capacity to let enter sodium ions into the cell. Any molecule either naturally occurring or synthetic, e.g., protein, oligopeptide, small organic molecule, polysaccharide, lipid (e.g. sphingolipid), fatty acid, polynucleotide, oligonucleotide, etc can be tested in the method of the invention. The potential modulator compound can be in the form of a library of compounds, such as a combinatorial or randomized library that provides a sufficient range of diversity.

In a preferred embodiment, the method comprises the following steps:
a) providing a cell of the invention expressing subunits of the ENaC or inducing their expression,
b) washing and subsequently loading the cell obtained in step a) with a fluorescent membrane potential dye,
c) contacting the cell obtained in step b) with a potential modulator compound in the presence of a cation and,
d) comparing cation-mediated changes in signal of the cell obtained in step c) with cation-mediated changes in signal of the cell obtained in c) in the absence of the potential modulator.

### Step a)

Depending on the type of expression system chosen, the skilled person may possibly adapt the culture conditions to obtain a most favourable expression level of ENaC subunits. In case of an inducible expression system, the skilled person may also possibly optimize the amount of inducing agent to be added to the cell. The time period of induction of the expression and the temperature during induction of the expression could also possibly be optimised. According to a preferred embodiment, at the onset of the induction of expression of ENaC subunits, sub-confluent cells are brought into a 96 well plate using a suitable culture medium. Sub-confluent preferably means about 70% confluent, more preferably about 80% confluent.

In a preferred embodiment, the inducing agent added is tetracycline or doxycyclin when using the preferred inducible expression system described above (tetracyclin-regulated promoter).

In a preferred embodiment, an inhibitor of the subunits of the epithelial sodium channel is further added. More preferably, the inhibitor is a known inhibitor of the human epithelial sodium channel. Even more preferably, this inhibitor is selected from the list consisting of: phenamil, benzamil and amiloride and any other derivatives thereof. Most preferably, the inhibitor is phenamil. Inhibiting the basal activity of the ENaC channel by adding an inhibitor is a way of reducing background activity of this channel and therefore improving the sensitivity of the assay.

### Step b)

At the end of step a), cells are washed and subsequently loaded with a fluorescent membrane potential dye. The washing medium is preferably iso-osmotic. More preferably, the washing medium is NMDG (N-methyl-D-glucamine) (Sigma). Membrane potential fluorescent dye is herein defined as a molecule or combination of molecules whose distribution changes upon membrane depolarization. These dyes can be used to detect the changes in activity of an ion channel such as ENaC when expressed in a cell. Any known membrane potential fluorescent dye may be used according to the manufacturer's instructions. Due to cost issue, the concentration could possibly be decreased without affecting its effectivity. Examples of membrane potential fluorescent dye are: Membrane Potential Assay kits from Molecular Devices, preferably MP-red (cat# R8123, R8126), or MP-blue (cat# R8042, R8034,), Di-4-ANEPPS (Pyridium, 4-(2-(6-(dibutylamino)-2-naphtalenyl)ethenyl)-1-(3-sulfopropyl))-hydroxide, inner salt), DiSBACC4(2) (bis-(1,2-dibarbituric acid)-trimethine oxanol), DiSBAC4(3) (bis-(1,3-dibarbituric acid)-trimethine oxanol), DiSBAC2(3) (bis-(1,3-diethylthiobarbituric acid)trimethine oxonol), CC-2-DMPE (Pacific Blue™ 1,2-dietradecanoyl-sn-glycerol-3-phosphoethanolmine, triethylammonium salt). Preferably, the membrane potential fluorescent dye is a Membrane Potential Assay kit MP-red or MP-blue from Molecular Devices as defined above.

### Step c)

Subsequently, the cells obtained in step b) are contacted with a potential modulator compound in the presence of a cation. The cation may be any cation known to be able to pass through the ENaC channel. Preferably, a suboptimal concentration of the cation is added to the cell. Preferably, the cation is sodium or lithium. More preferably, the cation is sodium. Even more preferably, a sub-optimal concentration of sodium or lithium is added to the cells. A sub-optimal concentration of sodium or lithium is preferably comprised between 10 and 100 mM, more preferably between 20 and 80 mM, even more preferably between 30 and 60 mM. Most preferably, the sub-optimal sodium or lithium concentration is about 50 Mm. Fluorescence is typically measured on a fluorescence plate reader. Preferably, when using a preferred Membrane Potential kit of Molecular Devices as defined before cell mixtures are excited at 530 nm. Fluorescence emission is measured at 565 nm.

### Step d)

The cation-mediated changes in signal of the cell obtained in step c) is compared with the cation-mediated changes in signal of the cell obtained in c) in the absence of the potential modulator. In other words, when a sub-optimal concentration of a cation has been added to the control cell (without potential modulator), typically a sub-maximal signal of the channel is observed. Compounds that increase the sub-optimal signal of the channel obtained with sub-optimal concentration of the cation, are potential enhancers of salty taste. In contrast, compounds that decrease the sub-optimal signal of the channel obtained with sub-optimal concentration of the cation, are potential maskers of salty taste. Sub-optimal concentrations of sodium or lithium have been defined in the paragraph dedicated to step c). Preferably, a sub-optimal signal of the channel is obtained using a sub-optimal concentration of sodium. Signal of the channel is herein defined as the change in relative fluorescence units of the cell measured after the addition of sodium ions.

According to a preferred embodiment, a potential enhancer of salty taste has been identified when the comparison performed in step d) indicates an increase of about at least 2% of sodium-mediated increase in signal. More preferably, a potential enhancer of salty taste has been identified when the comparison performed in step d) indicates an increase of about at least 4%, of about at least 6%, of about at least 8%, of about at least 10%, of about at least 12%, of about at least 14%, of about at least 16%, of about at least 18%, of about at least 20%, of about at least 22%, of about at least 24%, of about at least 26%, of about at least 28%, of about at least 30%, of about at least 32%, of about at least 34%, of about at least 36%, of about at least 38%, of about at least 40%, of about at least 42% or more, of sodium-mediated increase in signal. All these increases are preferably measured using a sub-optimal concentration of sodium, which is comprised between 10 and 100 mM, more preferably between 20 and 80 mM, even more preferably between 30 and 60 mM. Most preferably, the sub-optimal sodium concentration is about 50 Mm.

According to another preferred embodiment, a potential masker of salty taste has been identified when the comparison performed in step d) indicates a decrease of about at least 2% of sodium-mediated decrease in signal. More preferably, a potential masker of salty taste has been identified when the comparison performed in step d) indicates a decrease of about at least 4%, of about at least 6%, of about at least 8%, of about at least 10%, of about at least 12%, of about at least 14%, of about at least 16%, of about at least 18%, of about at least 20%, of about at least 22%, of about at least 24%, of about at least 26%, of about at least 28%, of about at least 30%, of about at least 32%, of about at least 34%, of about at least 36%, of about at least 38%, of about at least 40%, of about at least 42% or more of sodium-mediated decrease in signal. All these decreases are preferably measured using a sub-optimal concentration of sodium, which is comprised between 10 and 100 mM, more preferably between 20 and 80 mM, even more preferably between 30 and 60 mM. Most preferably, the sub-optimal sodium concentration is about 50 Mm.

According to a more preferred embodiment, the potential enhancer or masker of salty taste identified in step d) is further tested in the following steps comprising:
e) providing a cell of the invention expression the subunits or inducing their expression,
f) washing and subsequently loading the cell obtained in step a) with a sodium-sensitive fluorescent dye,
g) contacting the cell obtained in step f) with the potential enhancer or masker of salty taste as identified in step d) in the presence of sodium and,
h) comparing sodium-mediated changes in signal of the cell obtained in step g) with sodium-mediated changes in signal of the cell obtained in g) in the absence of the potential enhancer or masker of salty taste.

Step e) is similar with step a). Step f) is similar with step b) except that the fluorescent dye added is specific for sodium. Any commercial available sodium fluorescent dye may be used. Preferably the sodium fluorescent dye is selected from the list consisting of: SBFI (Sodium Binding benzofuran isophtalate), CoroNa-Green, CoroNa-Red, or Sodium-Green. All these dyes can be obtained from Molecular Probes. More preferably, the sodium fluorescent dye is SBFI.

An inhibitor of the ENaC channel is preferably used in step e) and/or f) as described in step a). Preferred inhibitors have already been defined in the paragraph relating to step a).

Step g) is similar to step c), with sodium as cation. The potential enhancer or masker identified in step d) is tested instead of the potential modulator compound.

Step h) is also similar to step d), with sodium as cation. The sodium-mediated changes in signal of the cell obtained in step g) are compared with the sodium-mediated changes in signal of the cell obtained in g) in the absence of the potential enhancer or masker identified in step d). In other words, when a sub-optimal concentration of sodium has been added to the control cell (without potential enhancer or masker), typically a sub-maximal signal of the channel is observed. In this step, a sub-optimal concentration of sodium is preferably comprised between 2 and 50 mM, more preferably between 5 and 40 mM, even more preferably between 7 and 30 mM, even more preferably between 10 and 25 mM.

Compounds that increase the sub-optimal signal of the channel obtained with a sub-optimal concentration of sodium, are potential enhancers of salty taste. In contrast, compounds that decrease the sub-optimal signal of the channel obtained with sub-optimal concentration of sodium, are potential masker of salty taste. Signal of the channel has already been defined earlier in the paragraph dedicated to step d). According to a preferred embodiment, a potential enhancer of salty taste has been identified when the comparison performed in step h) indicates an increase of about at least 2% of sodium-mediated increase in signal. More preferably, a potential enhancer of salty taste has been identified when the comparison performed in step h) indicates an increase of about at least 4%, of about at least 6%, of about at least 8%, of about at least 10%, of about at least 12%, of about at least 14%, of about at least 16%, of about at least 18%, of about at least 20%, of about at least 22%, of about at least 24%, of about at least 26%, of about at least 28%, of about at least 30%, of about at least 32%, of about at least 34%, of about at least 36%, of about at least 38%, of about at least 40%, of about at least 42% or more of sodium-mediated increase in signal. All these increases are preferably measured step using a sub-optimal concentration of sodium comprised between 2 and 50 mM, more preferably between 5 and 40 mM, even more preferably between 7 and 30 mM, even more preferably between 10 and 25 mM.

According to another preferred embodiment, a potential masker of salty taste has been identified when the comparison performed in step h) indicates a decrease of about at least 2% of sodium-mediated increase in signal. More preferably, a potential masker of salty taste has been identified when the comparison performed in step h) indicates a decrease of about at least 4%, of about at least 6%, of about at least 8%, of about at least 10%, of about at least 12%, of about at least 14%, of about at least 16%, of about at least 18%, of about at least 20%, of about at least 22%, of about at least 24%, of about at least 26%, of about at least 28%, of about at least 30%, of about at least 32%, of about at least 34%, of about at least 36%, of about at least 38%, of about at least 40%, of about at least 42% or more of sodium-mediated decrease in signal. All these decreases are preferably measured using a sub-optimal concentration of sodium, which is preferably comprised between 2 and 50 mM, more preferably between 5 and 40 mM, even more preferably between 7 and 30 mM, even more preferably between 10 and 25 mM.

A screening method combining these two assays is expected to be more efficient than a screening method using only one single assay.

As earlier mentioned herein, three types of host cells may be prepared:
expressing α, β and γ subunits or
expressing β, γ and δ subunits or
expressing α, β, γ and δ subunits all as earlier defined herein. It has been extensively demonstrated in the examples that these three types of cells are functional in the methods of the invention. Accordingly, in another preferred embodiment, the method of the invention comprising steps a) to d) or a) to h) is carried out using any of the three host cells of the invention. More preferably, the potential modulator compound tested in this method using one of the three cell types of the invention, is further tested in this method using another cell type of the invention. It is also encompassed to subsequently test a potential modulator compound in each cell type of the invention. The use of one to three distinct cell types of the invention in the method of the invention may lead to additional information as to the characteristics of the potential modulator compound.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The invention is further described by the following examples, which should not be construed as limiting the scope of the invention.

### EXAMPLES

### Example 1

### Functional Mammalian Cells Expressing the Alpha Beta and Gamma Chains of the Human Epithelial Sodium Channel

### 1. Description of the Expression Construct Used

Recombinant plasmids for the transfection of mammalian cell lines were constructed using standard molecular biological techniques. FIG. 1 shows a typical example of a nucleotide vector used for transfection of mammalian cell lines. The vector is based on pcDNA5/FRT/TO (Invitrogen) and contains the coding DNA sequences for three subunits of the human epithelial sodium channel arranged in a head to tail orientation (SEQ ID NO:17 coding for alpha, SEQ ID NO:19 coding for beta, and SEQ ID NO:21 coding for gamma). Each of them was preceded with a tetracycline-inducible promoter. The plasmid contained an FRT (Flp Recombination Target) site, which allows the site-specific integration of the plasmid DNA via Flp recombinase-mediated DNA recombination at the FRT site of a mammalian cell line having a FRT site in his genome.

No efficient commercial antibodies exist for the gamma subunit. Therefore, in order to investigate whether the γ subunit was produced in the cells, a derivative of pUR8191 was created in which the γ subunit was replaced by a V5-tagged version (γ-V5-tag), using standard recombinant DNA techniques. Antibodies against a V5-tag were commercially obtained from Invitrogen.

### 2. Transformation of Cells

Human cells Flp-In T-Rex 293 (Invitrogen, cat# R780-07) were transfected using Lipofectamine 2000 (Invitrogen). The transfections were carried out essentially according to the manufacturer's manual. In a 12-well plate, 0.5×10⁶ cells per well were seeded in 1 ml DMEM (Life Technologies 31966-021)/10% FCS without antibiotics. The next day, 2 μg of the expression vector DNA prepared in 1) and 8 μg of pOG44 DNA (expressing the Flp-recombinase) were added into 100 μl Opti-MEM. Subsequently, 4 μl Lipofectamine 2000 was added into these 100 μl Opti-MEM. This mixture was incubated for 30 minutes and added to the cells. After 1 to 2 days, the medium was refreshed by adding DMEM medium supplemented with 10% tetracyclin-free FCS (BD Biosciences 631101 or 631106) containing 5 μg/ml blasticidin and 100 μg/ml hygromycin. About every 2-3 days the medium was refreshed until surviving cell lines were clearly visible by eye. When clearly visible, clones were transferred to an appropriate volume (96-well-6-well plate) and subsequently screened for production of the subunit proteins.

### 3. Evidence of Expression of the Channel Subunits

For protein analysis of the alpha, beta and gamma subunits, a 6-well plate was inoculated with 1.5×10⁶ stable HEK293 T-Rex Flp-In hENaC cells in 2 ml medium per well and grown for about 20 hrs.

hENaC channel expression was induced by addition of 0.6 ml DMEM growth medium containing doxycyclin (final conc. 0.25 μg/ml) per well. After 20-48 hrs, the cells were scraped off, centrifuged for 2 min. at 2000 rpm, washed with 1 ml PBS and resuspended in 90 μl PBS with Complete EDTA (Roche, 1873580, 1 tablet per 10 ml) to inhibit protease activity. Cells were disrupted by sonication with a Soniprep 150 (MSE) for 30 sec with an amplitude of 6 microns, placed on ice and frozen upon usage. The protein concentration in the sample was determined using a BioRad protein determination kit (5 μl sample, 40 μl BioRad reagent and 160 μl water) according to the manufacturer's protocol. Before gel loading, 30 μl of 4× sample buffer (8% SDS, 40% glycerol, 0.01% Bromophenol Blue, 0.2 M Tris-HCl pH 6.8, 0 mercapto-ethanol 75 μl/ml) was added.

About 10 μg per sample was separated by SDS-PAGE on a 8% Novex Tris-glycine gel (Invitrogen, cat. EC6015) at 125 V. For western analysis, the proteins were transferred onto an Immobilon polyvinylidene-difluoride membrane (Millipore, cat. no IPVH00010) by the Ancos semi dry blotting method (150 mA, 1.5 hr for 2 blots). Unspecific staining on the blot was blocked in PBS containing 4% (w/v) dry milk powder (Marvel) for 15 min. The PBS/Marvel was removed and the blot was incubated overnight with the first antibody, being either anti-α (1:2500 dilution, ABR PA 1-920), anti-β (1:2500 dilution, ABR PA1-921) or anti-V5 (1:2000 dilution, Invitrogen) in PBS/0.1% Marvel.

The blot was subsequently washed three times with 50 ml PBS-T (contains 0.05% v/v Tween 20) and incubated with a 1:5000 dilution of an anti-rabbit AP conjugate (Promega, S3731) for α and β or a 1:5000 dilution of an anti-mouse AP conjugate (Promega, S3721) for the V5-tag for 2 hours. After washing three times with PBS-T the proteins were visualised with the alkaline phosphatase conjugate substrate kit of Biorad (NBT/BCIP).

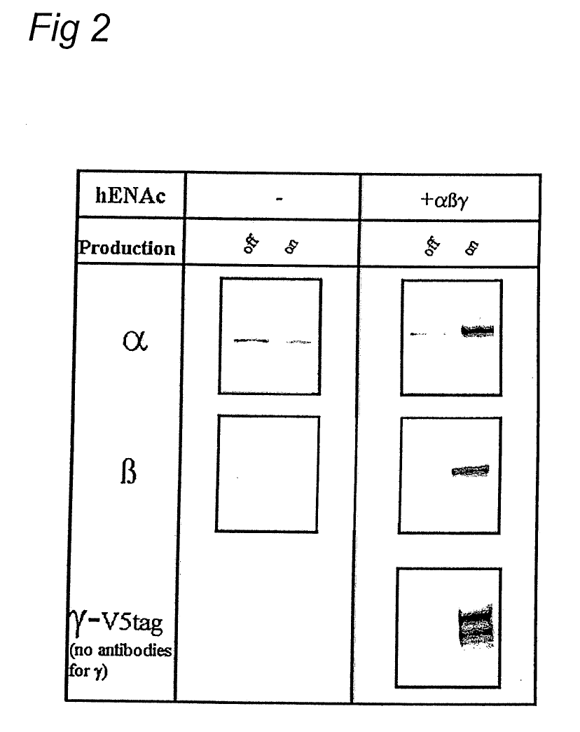

All three subunits were produced in high amounts in induced cells whereas in non-induced cells, no bands were observed except for the anti-α antibody that showed a faint band that was slightly smaller in size than the band observed for the α subunit in the induced cells (see FIG. 2). This band probably represents a protein that is cross-reacting with the antibody, as it is also present in wild type cells. The antibodies anti-β and anti-V5 only recognised proteins in the induced cell lines, with a molecular weight expected for the β and γ subunit. The anti-V5 antibody recognised multiple bands, which probably represent different forms of glycosylation, as all subunits are also glycosylated in their extracellular domain in human cells.

### 4. Primary Screen: MP Assay

HEK293 Flp-In T-Rex hENaC cells were grown to about 80% confluency and dissociated from the culture dish with trypsin/EDTA for 1-5 minutes at 37° C. Dissociated cells were taken up in a concentration between 1×10⁵ and 1×10⁶ cells/ml in DMEM/Tet-free FCS/blasticidin/hygromycin medium. 100 μL of cells were added per well in a poly-L-lysine coated black well clear bottom 96-wells plate. The cells were grown overnight and then induced by addition of 100 μl DMEM/Tet-free FCS/blasticidin/hygromycin medium with doxycyclin (final conc. 0.25 μg/ml). Alternatively, in some wells amiloride was added to inhibit hENaC channel activity during induction.

After 20 to 48 hrs the medium was removed, cells were washed with buffer and subsequently loaded with MP-Blue (cat# R8042) or MP-Red (cat# R8123) dye (from Molecular Devices) according to the manufacturer's instructions for 1 hr at 37° C. The plates were subsequently loaded into the Flexstation II for fluorescence measurements. Compounds were added to the wells in different concentrations after a baseline measurement of 30 sec. Measurements were carried out at 37° C. with a 4-sec interval for 300 sec and excitation/emission wavelength 530/565 nm. The fluorescence values were normalized on the average value of the first five time points.

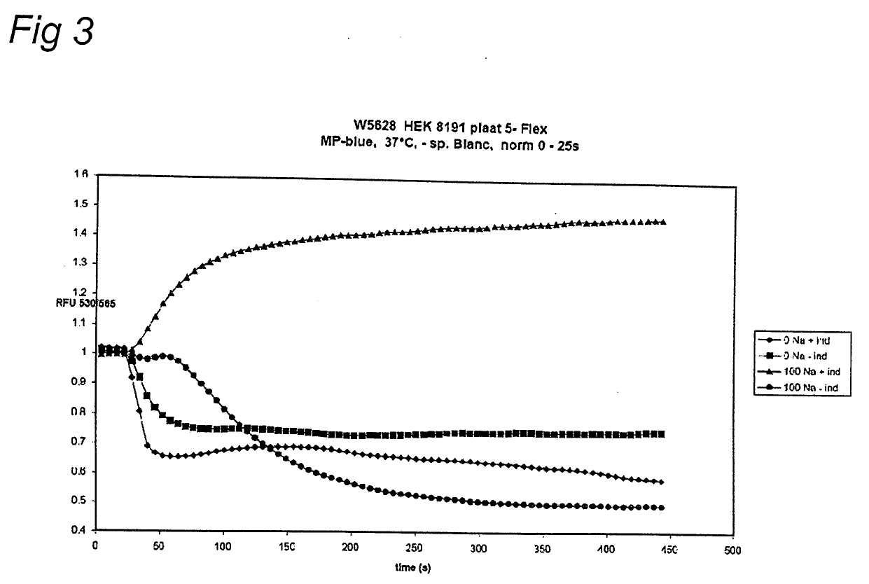

As an example of the sodium-dependent response of HEK293-Flp-In T-Rex hENaC cells with the membrane potential dye, induced and non-induced cells were loaded with MP-Blue as described above and stimulated with 0 and 100 mM NaCl. As shown in FIG. 3, only the hENaC expressing cells (induced cells) displayed an increase in fluorescence signal after addition of the Na⁺-buffer, indicative of stronger membrane depolarisation due to increased Na⁺-influx. Addition of NMDG (N-methyl-D-glucamine) from Sigma leads in hENaC-expressing cells to a hyperpolarisation, visible as a reduction in fluorescence signal. This hyperpolarisation is caused by an efflux of Na⁺ ions, since the intracellular Na⁺ concentration is higher than the extracellular concentration. Stimulation of non-induced cells with either 100 mM NaCl or with buffer without NaCl also evokes a hyperpolarisation. This hyperpolarisation, however, is most probably caused by the efflux of K⁺-ions, since, due to the absence of Na⁺ channels, in these cells the K⁺ channels are the major determinant for the membrane potential and the K⁺ concentration inside the cells is higher than outside.

### Role of ENaC Inhibitors

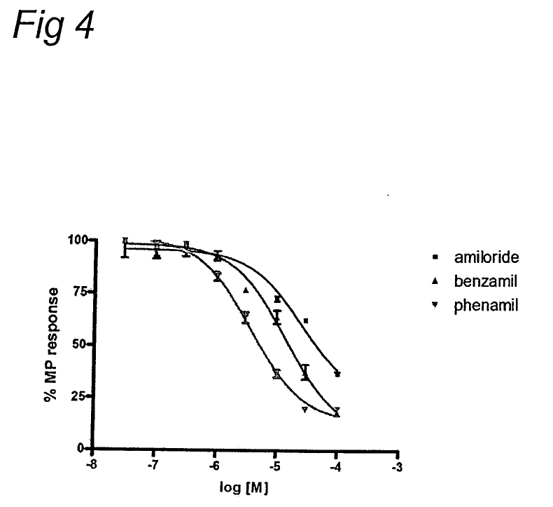

To verify that the hENaC channel can be blocked by the diuretic molecule amiloride or its derivatives benzamil and phenamil, dose-response curves for inhibition were generated, according to the method described above. As shown in FIG. 4, the best compound for inhibition is phenamil, followed by benzamil and amiloride.

### 5. Secondary Screen: SBFI Assay

HEK293 Flp-In T-Rex hENaC cells were grown to about 80% confluency and dissociated from the culture dish with trypsin/EDTA for 1-5 minutes at 37° C. Dissociated cells were taken up in a concentration between 1×10⁵ and 1×10⁶ cells/ml in DMEM/Tet-free FCS/blasticidin/hygromycin medium. 100 μl of cells was added per well in a poly-L-lysine coated black well clear bottom 96-wells plate. The cells were grown overnight and then induced by addition of 100 μl DMEM/Tet-free FCS/blasticidin/hygromycin medium with doxycyclin (final conc. 0.25 μg/ml). Alternatively, amiloride was added in some wells to inhibit hENaC channel activity during induction.

After 20-48 hrs, the medium was removed, the cells were washed once, loaded with SBFI (Molecular Probes) in dye loading buffer according to the manufacturer's instructions and incubated at 37° C. for 1 hr. Then, the dye was removed, the cells were washed once and taken up in 70 μl buffer.

The plate was then loaded into a Fluorescence Plate reader (Flexstation II, Molecular Devices) for determination of the fluorescence levels at the excitation-emission wavelength pairs 340/510 nm and 380/510 nm. After determination of the baseline, compounds were added, typically 100 mM NaCl or buffer (no NaCl), or a concentration range of NaCl and the normalised 340/380 nm ratio was determined.

FIG. 5 shows an example of such an experiment with 100 mM NaCl added to either induced or non-induced cells. This experiment confirms the functionality of SBFI-based screen in the cells of the invention.

### Example 2

### Functional Mammalian Cells Expressing Either the Beta, Gamma and Delta Chains or the Alpha, Beta, Gamma and Delta Chains of the Human Epithelial Sodium Channel

The same strategy was used as in example 1. The vectors used were similar to the ones of example 1 except for the identity of the nucleic acid sequences present. The vectors are depicted in FIGS. 6, 7. As nucleic acid sequence coding for the beta, gamma and delta chains of the human epithelial sodium channel, the following nucleic acid sequences were used: SEQ ID NO: 19, SEQ ID NO: 21 and SEQ ID NO: 23 respectively.

As nucleic acid sequence coding for the alpha, beta, gamma and delta chains of the human epithelial sodium channel, the following nucleic acid sequences were used: SEQ ID NO: 17, SEQ ID NO: 19, SEQ ID NO: 21 and SEQ ID NO: 23 respectively. The cells used were the same as in example 1.

To demonstrate the functionality of both cells lines expressing either the beta, gamma and delta chains or the alpha, beta, gamma and delta chains of the human epithelial sodium channel in the screening methods of the invention, the primary screen (MP assay) was carried out as in example 1 (FIGS. 8, 9). Results obtained were similar to the ones obtained in FIG. 3. Subsequently, the secondary screen (SBFI assay) was carried out as in example 1 (FIGS. 10, 11). Results obtained were similar to the ones obtained in FIG. 5. Both screening assays were functional in both cell lines.

1. A nucleic acid molecule being represented by a nucleic acid sequence said nucleic acid sequence comprising at least three nucleic acid subsequences arranged in a head to tail configuration with respect to another, wherein two of these nucleic acid subsequences code for the β and γ subunits of the epithelial sodium channel, and additional nucleic acid subsequence(s) is(are) selected from the group consisting of: a nucleic acid subsequence coding for the α subunit and a nucleic acid subsequence coding for the δ subunit of the epithelial sodium channel and wherein each encoded subunit of the epithelial sodium channel has an amino acid sequence that has about 85% or more amino acid sequence identity with the amino acid sequence encoded by the corresponding nucleic acid subsequence having the following SEQ ID NO:
SEQ ID NO:19 for the β subunit, and
SEQ ID NO:21 for the γ subunit, and
SEQ ID NO:17 for the α subunit, and/or
SEQ ID NO:23 for the δ subunit respectively.

2. The nucleic acid molecule according to claim 1, wherein the expression of each nucleic acid subsequence is inducible.

3. The nucleic acid molecule according to claim 2, wherein the expression of each nucleic acid subsequence is inducible due to the presence of an inducible promoter operably linked to each of these nucleic acid subsequence.

4. A nucleic acid construct comprising the nucleic acid sequence as defined in claim 1.

5. The nucleic acid construct according to claim 4, wherein each nucleic acid subsequence is operably linked to one or more additional control sequences, which direct the production of the subunits in a suitable expression host.

6. An expression vector comprising the nucleic acid construct of claim 5 and transcriptional and translational stop signals.

7. A host cell comprising the nucleic acid construct of claim 4 or 5 or the expression vector of claim 6.

8. An in vitro method for screening a potential modulator compound of the subunits of the epithelial sodium channel using the cell of claim 7.

9. The method according to claim 8 comprising:
a) providing a cell as defined in claim 7 expressing the subunits or inducing their expression,
b) washing and subsequently loading the cell obtained in step a) with a fluorescent membrane potential dye,
c) contacting the cell obtained in step b) with a potential modulator compound in the presence of a cation and,
d) comparing cation-mediated changes in signal of the cell obtained in step c) with cation-mediated changes in signal of the cell obtained in c) in the absence of the potential modulator.

10. The method according to claim 9, wherein in step a) an inhibitor of the epithelial sodium channel is added.

11. The method according to claim 9, wherein the membrane potential fluorescent dye is selected from the list consisting of: Membrane Potential Assay kits from Molecular Devices, preferably MP-red (cat# R8123, R8126), or MP-blue (cat# R8042, R8034,), Di-4-ANEPPS (Pyridium, 4-(2-(6-(dibutylamino)-2-naphtalenyl)ethenyl)-1-(3-sulfopropyl))-, hydroxide, inner salt), DiSBACC4(2) (bis-(1,2-dibarbituric acid)-trimethine oxanol), DiSBAC4(3) (bis-(1,3-dibarbituric acid)-trimethine oxanol), DiSBAC2(3) (bis-(1,3-diethylthiobarbituric acid)trimethine oxonol), and CC-2-DMPE (Pacific Blue™ 1,2-dietradecanoyl-sn-glycerol-3-phosphoethanolmine, triethylammonium salt).

12. The method according to claim 9, wherein the cation is sodium.

13. The method according to claim 12, wherein a potential enhancer of salty taste has been identified when the comparison performed in step d) indicates an increase of at least 2% of sodium-mediated increase in signal.

14. The method according to claim 12, wherein a potential masker of salty taste has been identified when the comparison performed in step d) indicates a decrease of at least 2% of sodium-mediated decrease in signal.

15. The method according to claim 13 or 14, wherein the potential enhancer or masker of salty taste identified in step d) is further tested in the following steps comprising:
e) providing a cell as defined in claim 7 expressing the subunits or inducing their expression,
f) washing and subsequently loading the cell obtained in step e) with a sodium-sensitive fluorescent dye,
g) contacting the cell obtained in step f) with the potential enhancer or masker of salty taste as identified in step d) in the presence of sodium and,
h) comparing sodium-mediated changes in signal of the cell obtained in step g) with sodium-mediated changes in signal of the cell obtained in g) in the absence of the potential enhancer or masker of salty taste.

16. The method according to claim 15, wherein in step e) and/or f) an inhibitor of the epithelial sodium channel is added.

17. The method according to claim 16, wherein the sodium-sensitive fluorescent dye is selected from the list consisting of: SBFI, CoroNa Green, CoroNa-Red, and Sodium Green.

18. The method according to claim 17, wherein an enhancer of salty taste has been identified when the comparison performed in step h) indicates an increase of at least 2% of sodium-mediated increase in signal.

19. The method according to claim 17, wherein a masker of salty taste has been identified when the comparison performed in step h) indicates a decrease of at least 2% of sodium-mediated decrease in signal.
